(19) United States
(12) Patent Application Publication
(10) Pub. No.: US 2026/0167974 A1
(43) Pub. Date: Jun. 18, 2026

(54) Novel Methods for Treating Glaucoma

(71) Applicant: The Regents of the University of California, Oakland, CA (US)

(72) Inventor: Lu Chen, Berkeley, CA (US)

(73) Assignee: The Regents of the University of California, Oakland, CA (US)

(21) Appl. No.: 19/302,120

(22) Filed: Aug. 18, 2025

Related U.S. Application Data
(63) Continuation of application No. PCT/US24/17149, filed on Feb. 23, 2024.
(60) Provisional application No. 63/486,665, filed on Feb. 23, 2023.

(51) Int. Cl. C12N 15/113; A61K 9/00; A61P 27/06

(52) U.S. Cl. CPC C12N 15/1138; A61K 9/0048; A61P 27/06; C12N 2310/14; C12N 2310/141

(57) ABSTRACT

The present disclosure generally relates to treatment of glaucoma and/or the reduction of elevated intraocular pressure (IOP). In particular, the present disclosure relates to compositions and methods for preventing or treating glaucoma, including primary glaucoma, by inhibiting factors associated with ocular lymphatic formation.

### CROSS REFERENCE TO RELATED APPLICATIONS

This application is a continuation of PCT/US24/17149, filed: Feb. 23, 2024, which claims priority to Ser. 63/486,665, filed Feb. 23, 2023. The entirety of these priority applications is hereby incorporated by reference herein for all purposes.

### STATEMENT OF FEDERALLY FUNDED RESEARCH

This invention was made with Government support under Grant Nos. EY028995 and EY017392, awarded by the National Institutes of Health. The Government has certain rights in this invention.

### FIELD OF THE INVENTION

The present disclosure generally relates to treatment or prevention of glaucoma and/or the reduction of elevated intraocular pressure (IOP). In particular, the present disclosure relates to compositions and methods for preventing or treating glaucoma, and in particular a primary glaucoma, by inhibiting factors associated with lymphatic formation and the Schlemm's canal in the eye.

### INCORPORATION BY REFERENCE

The contents of the XML file named “23091-006WO1_ST26_2024-02-19.xml” which was created on Aug. 17, 2025, and is 44,781 bytes in size, are hereby incorporated by reference in their entirety.

### BACKGROUND OF THE INVENTION

Glaucoma is a group of eye diseases that result in damage to the optic nerve. If left untreated, glaucoma can lead to vision loss and even blindness. About 80 million people worldwide have glaucoma, with about three million of those in the United States.

Glaucoma can be classified as primary or secondary. Primary glaucoma is hallmarked by an increased resistance to aqueous outflow, resulting in increased intraocular pressure (IOP) and ultimately optic nerve damage, and the absence of other causative underlying diseases. Primary open angle glaucoma (POAG) is a subset of glaucoma defined by an open, normal appearing anterior chamber angle and raised intraocular pressure (IOP), with no other underlying disease. Comparatively, secondary glaucoma is generally caused by another medical condition. One particular secondary glaucoma is neovascular glaucoma (NVG) which is characterized by proliferation of fibrovascular tissue in the anterior chamber angle (see Wand M., Neovascular glaucoma. In: Ritch R, Shields M B, Krupin T, eds. The Glaucomas—Clinical Science. 2^{nd}. Ed. St. Louis: Mosby; 1996:1073-1129. Ch 51). The common denominator predisposing to NVG is usually retinal ischemia, although some cases are associated with other ocular or extraocular entities.

Eye pressure is measured in millimeters of mercury (mm Hg). Normal eye pressure ranges from 12-21 mm Hg in human, and eye pressure of greater than 21 mm Hg is considered higher than normal or elevated. Elevated IOP in primary glaucoma is chiefly caused by dysregulated drainage of the aqueous humor (the clear fluid that fills the front of the eye). The Schlemm's canal is a critical structure by which aqueous humor drainage and IOP is maintained. The Schlemm's canal is a core component of the conventional aqueous humor outflow pathway, which accounts for 70-90% of the total aqueous humor outflow that drains from the human eye. Furthermore, the endothelial cell lining of the Schlemm's canal is one of the primary sites of resistance to aqueous humor drainage and is a major determinant of IOP (Mäepea, O. & Bill, A. Pressures in the juxtacanalicular tissue and Schlemm's canal in monkeys. Exp Eye Res. 54 (6): 879-883 (1992 June)). The Schlemm's canal in glaucomatous human eyes shrinks significantly compared to healthy, normal eyes (Gabelt, B. T. & Kaufman, P. L. Changes in aqueous humor dynamics with age and glaucoma. Prog Retin Eye Res. 24:612-637 (2005)). A resultant resistance caused by Schlemm's canal shrinkage and other morphological changes increases with age or under a pathological situation, causing IOP to be elevated. The abnormally elevated IOP then leads to optic nerve damage and vision loss. This is exemplified by the fact that Schlemm's canal shrinkage alone accounts for approximately 50% of the loss of total outflow facility observed in primary open angle glaucoma (POAG) eyes (Allingham, R. R. et al. Schlemm's canal and primary open angle glaucoma: correlation between Schlemm's canal dimensions and outflow facility. Exp. Eye Res. 62:101-109 (1996)). Moreover, the density of Schlemm's canal inner wall pores is decreased five-fold in glaucomatous eyes compared to normal eyes (Johnson, M. et al. The pore density in the inner wall endothelium of Schlemm's canal of glaucomatous eyes. IOVS. 43:2950-2955 (2002)). The Schlemm's canal is therefore a key ocular structure involved in the pathogenesis of glaucoma. The particular mechanisms by which the Schlemm's canal regulates aqueous humor outflow in both normal and glaucomatous eyes, however, still remain elusive.

Some studies suggest that the lymphangiogenesis pathway is a possible mechanism that may play a role in the pathogenesis of glaucoma. Lymphangiogenesis is the process of the formation of new lymphatic vessels from pre-existing vessels or lymphatic endothelial precursor cells. For example, researchers have found that genetic disruption of lymphatic vessels may contribute to elevated IOP. Accordingly, it had been previously proposed that inducing or activating lymphangiogenesis may potentially treat glaucoma (see, e.g., Aspelund, A. et al. The Schlemm's canal is a VEGF-C/VEGFR-3-responsive lymphatic-like vessel. J Clin Invest. 124 (9): 3975-3986 (2014 September); Thomson, B. R. et al. A lymphatic defect causes ocular hypertension and glaucoma in mice. J Clin Invest. 124 (10): 4320-4324 (2014 October; Epub 2014 Sep. 9); Kim, J. et al. Impaired angiopoietin/Tie2 signaling compromises Schlemm's canal integrity and induces glaucoma. J Clin Invest. 127 (10): 3877-3896 (2017 Oct. 2; Epub 2017 Sep. 18)).

Early treatment of glaucoma is possible to slow or stop the progression of the disease. The goal of the treatment is to lower elevated IOP and protect the optic nerve from damage. The most common treatments for glaucoma include eye drops that decrease the amount of fluid produced in the eye or improve the drainage of fluid from the eye via the uveoscleral pathway, oral medication that lower IOP, laser therapy (e.g., laser trabeculoplasty and laser iridotomy), and surgery (e.g., implants, trabeculectomy, canaloplasty). One such surgical procedure, canaloplasty, seeks to lower IOP by catheterizing and dilating the Schlemm's canal using a microcatheter that also introduces a suture. The suture provides circumferential tension to the inner wall Schlemm's canal so that the canal expands, which has proven to effectively increase aqueous humor outflow and decrease IOP (Lewis, R. A. et al. Canaloplasty: Three-year results of circumferential viscodilation and tensioning of Schlemm canal using a microcatheter to treat open-angle glaucoma. J Cataract Refract Surg. 37 (4): 682-690 (2011 April)). Several complications are associated with canaloplasty, however, which include microhyphema (12.1%), hypotony (0.6%), and the common post-procedure observation of blood in the anterior chamber due to the rapid IOP decrease (Id.). The current glaucoma treatment options, therefore, have limited efficacy and are associated with side effects or complications.

Therefore, there is an urgent need to develop new therapies for the treatment of glaucoma, including a primary glaucoma.

### SUMMARY OF THE INVENTION

It has been surprisingly discovered that inhibiting lymphangiogenesis factors which mediate lymphatic formation or maintenance (“pro-lymphatic formation factors”) and are associated with the Schlemm's canal in the eye effectively lowers intraocular pressure (IOP) and provides an effective treatment for glaucoma. The lymphatic system regulates body fluid balance and immune function, with lymphatic vessels transporting lymphatic fluid containing immune cells and other components. The eye comprises a mixture of tissues that are alternatively rich in lymphatic vessels or devoid thereof (Chen, L. Ocular lymphatics: state-of-the-art review. Lymphology. 42 (2): 66-76 (2009 June)). As reported herein, targeting pro-lymphangiogenic formation factors associated with the Schlemm's canal in the eye, for example, but not limited to major pro-lymphatic formation factors such as VEGFR-3, VLA-1, Ang-2, or ITGA5, significantly lowered IOP in a mouse model of glaucoma (see, e.g., FIGS. 1A, 2A, 3A, 4A, and 5A). This finding is in direct contradiction to previous suggestions in the art that reductions in elevated IOP, and thus the treatment of glaucoma, including a primary glaucoma, could be achieved by stimulating lymphangiogenesis.

It is highly unexpected that inhibiting pro-lymphatic formation factors which are associated with the Schlemm's canal not only successfully reduces IOP but also mitigates several hallmark conditions associated with glaucoma. It was not previously known if and to what extent lymphangiogenesis regulates the progression or severity of complex ocular disorders such as primary glaucoma. Unlike most organs in the body which are normally penetrated by lymphatic vessels, the eye comprises a heterogeneous collection of tissues that are either lymphatic-rich (e.g., conjunctiva) or fully devoid of lymphatic vessels (e.g., cornea) under normal physiological conditions (Chen, L. Ocular lymphatics: state-of-the-art review. Lymphology. 42 (2): 66-76 (2009 June)). Moreover, some ocular tissues can be induced to form lymphatic vessels (e.g., cornea), while others (e.g., limbal lymphatics) harbor classical lymphatic-specific markers such as LYVE-1 which is absent in the Schlemm's canal (Id.). Accordingly, the prevailing molecular or mechanistic strategies in the art have been to generate new lymphatic vessels by activating lymphangiogenesis in response to or in an effort to reduce elevated IOP (see, e.g., WO2015110701A1; Clahsen, T. et al. The novel role of lymphatic vessels in the pathogenesis of ocular diseases. Prog Retin Eye Res. 96: 101157 (2023 September; Epub 2023 Feb. 8); Aspelund et al. 2014, The Schlemm's canal is a VEGF-C/VEGFR-3-responsive lymphatic vessel. JCI 124 (9); 2014:3975-3986; Thomson et al. A lymphatic defect causes ocular hypertension and glaucoma in mice. JCI 124 (10); 2014:4320-4324; Kim et al., Impaired angiopoietic/Tie-2 signaling compromises Schlemm's canal integrity and induces glaucoma. JCI 127 (10); 2017:3877-3896). To the contrary, however, as described herein it has been discovered that administering an inhibitor of a pro-lymphatic formation factor that induces or maintains lymphangiogenesis results in the reduction of IOP and several hallmarks of glaucomatous damage, including corneal edema (see, e.g., FIGS. 1B and 2B), retinal nerve fiber layer thinning (see, e.g., FIG. 2C), and retinal ganglion cell (RGC) death (see, e.g., FIG. 2D).

As shown herein, the inhibition of pro-lymphatic formation factors somewhat mimics surgical procedures, such as canaloplasty, to target and enhance the drainage function of pre-existing lymphatic and lymphatic-like structures (e.g., the Schlemm's canal). Administering an inhibitor of a pro-lymphatic formation factor to the eye increases the permeability of the structure, which allows for increased aqueous outflow and a lowering of elevated IOP (see, e.g., FIGS. 1C, 3B-3C, 4B-4C, and 5B). The Schlemm's canal is a critical structure in the regulation of aqueous humor drainage and IOP as a crucial component of the conventional outflow pathway. Although the Schlemm's canal has been suggested to play a role in glaucoma, it was not previously understood whether activating or inhibiting particular lymphatic mechanisms in the Schlemm's canal could prevent or treat glaucoma, especially primary glaucoma. Unexpectedly, it has been discovered that molecularly inhibiting a pro-lymphatic formation factor surprisingly can treat glaucoma, including primary glaucoma, by inhibiting Schlemm's canal cell function. For example, inhibition of the pro-lymphatic formation factors VEGFR-3, VLA-1, Ang-2, or ITGA5 reduces Schlemm's canal endothelial cell adhesion (see, e.g., FIGS. 1C, 3B, 4B, and 5B) and reduces Schlemm's canal tube formation (see, e.g., FIGS. 3C and 4C).

While not wanting to be bound to any one theory, it is proposed that inhibiting pro-lymphatic formation factors targets certain functions of the Schlemm's canal to increase the permeability thereof, which contribute to enhanced aqueous humor drainage via the Schlemm's canal that provides measurable decreases in IOP and a reduction in glaucomatous damage. Furthermore, while again not wishing to bound by any one theory, the reduced Schlemm's canal cell adhesion and tube formation contributes to increased Schlemm's canal permeability in vivo to allow increased outflow facility and provides a reduction in IOP and alleviation of several parameters of glaucomatous damage.

The discovery described herein provides an advance in glaucoma therapy by providing methods for preventing or treating glaucoma, including a primary glaucoma such as primary open-angle glaucoma (POAG), primary angle-closure glaucoma (PACG), primary congenital glaucoma, and primary normal tension glaucoma, as well as other IOP-mediated ocular disorders through the inhibition of a major pro-lymphangiogenesis factor.

In one aspect, the present disclosure provides a method for preventing or treating glaucoma in an eye of a subject in need thereof, the method comprising administering to the eye a therapeutically effective amount of an inhibitor of pro-lymphatic formation factor. In some embodiments, the glaucoma is a primary glaucoma selected from POAG, PACG, primary congenital glaucoma, and primary normal tension glaucoma.

In one aspect, the present disclosure provides a method of inhibiting lymphangiogenesis in an eye of a subject suffering from an elevated IOP, the method comprising administering to the eye a therapeutically effective amount of an inhibitor of a pro-lymphatic formation factor. In some embodiments, the subject has an elevated IOP of greater than about 21 mm Hg. In some embodiments, the subject has an elevated IOP of greater than about 25 mm Hg. In some embodiments, the subject has an elevated IOP of greater than about 30 mm Hg. In some embodiments, following administration of the inhibitor, the subject's IOP in the treated eye is reduced by at least about 1 mm Hg, at least about 2 mm Hg, at least about 5 mm Hg, at least about 10 mm Hg, or at least about 15 mm Hg.

In another aspect, the present disclosure provides a method of inhibiting lymphangiogenesis in an eye of a subject having or suspected of having normal tension glaucoma, the method comprising administering to the eye a therapeutically effective amount of an inhibitor of a pro-lymphatic formation factor.

In one aspect, the present disclosure provides a method of inhibiting lymphangiogenesis in an eye of a subject at risk for the development of glaucoma, the method comprising administering to the eye a therapeutically effective amount of an inhibitor of a pro-lymphatic formation factor. In some embodiments, the glaucoma is a primary glaucoma selected from POAG, PACG, primary congenital glaucoma, and primary normal tension glaucoma.

In one aspect, the present disclosure provides a method for increasing Schlemm's canal permeability in an eye of a subject suffering from an elevated IOP, the method comprising administering to the eye a therapeutically effective amount of an inhibitor of a pro-lymphatic formation factor, thereby increasing Schlemm's canal permeability, increasing aqueous humor outflow, and lowering the IOP of the eye. In some embodiments, the subject has an elevated IOP of greater than about 21 mm Hg. In some embodiments, the subject has an elevated IOP of greater than about 25 mm Hg. In some embodiments, the subject has an elevated IOP of greater than about 30 mm Hg. In some embodiments, following administration of the inhibitor, the subject's IOP in the treated eye is reduced by at least about 1 mm Hg, at least about 2 mm Hg, at least about 5 mm Hg, at least about 10 mm Hg, or at least about 15 mm Hg.

In another aspect, the present disclosure provides a method for increasing Schlemm's canal permeability in an eye of a subject having or suspected of having normal tension glaucoma, the method comprising administering to the eye a therapeutically effective amount of an inhibitor of a pro-lymphatic formation factor, thereby increasing Schlemm's canal permeability, increasing aqueous humor outflow, and lowering the IOP of the eye.

In one aspect, the present disclosure provides a method for modulating one or more physiological functions of the Schlemm's canal in the eye, the method comprising administering to the eye a therapeutically effective amount of an inhibitor of a pro-lymphatic formation factor, thereby modulating one or more physiological functions of the Schlemm's canal. In some embodiments, the physiological function modulated includes inhibiting Schlemm's canal cell adhesion, proliferation, migration, or tube formation.

In one aspect, the present disclosure provides a method for increasing aqueous humor outflow of the Schlemm's canal from the anterior chamber in an eye of a subject in need thereof, the method comprising administering to the eye a therapeutically effective amount of an inhibitor of a pro-lymphatic formation factor, thereby increasing aqueous humor outflow of the Schlemm's canal.

In one aspect, the present disclosure provides a method for reducing corneal edema in an eye of a subject in need thereof, the method comprising administering to the eye a therapeutically effective amount of an inhibitor of a pro-lymphatic formation factor, thereby reducing corneal edema.

In one aspect, the present disclosure provides a method for reducing retinal nerve fiber layer thinning in an eye of a subject in need thereof, the method comprising administering to the eye a therapeutically effective amount of an inhibitor of a pro-lymphatic formation factor thereby reducing retinal nerve fiber layer thinning.

In one aspect, the present disclosure provides a method for reducing retinal ganglion cell (RGC) death in an eye of a subject in need thereof, the method comprising administering to the eye a therapeutically effective amount of an inhibitor of a pro-lymphatic formation factor, thereby reducing RGC death.

As provided herein, the methods provide administering to an eye of a subject in need thereof an inhibitor of a pro-lymphatic formation factor. In some embodiments, the pro-lymphatic formation factor is associated with the Schlemm's canal. In some embodiments, the pro-lymphatic formation factor is a factor which induces lymphangiogenesis. In some embodiments, the pro-lymphatic formation factor is a factor which maintains or patterns lymphangiogenesis. In an alternative embodiment, the inhibitor of lymphangiogenesis can be an agent that activates or stimulates an anti-lymphatic formation factor, as described further below.

In some embodiments, the pro-lymphatic formation factor targeted for inhibition is a vascular endothelial growth factor/vascular endothelial growth factor receptor (VEGF/VEGFR) family member. In some embodiments, the VEGF/VEGFR family member is selected from the group consisting of VEGF-A, VEGF-B, VEGF-C, VEGF-D, VEGF-E, PIGF, VEGFR-1, VEGFR-2, and VEGFR-3. In some embodiments, the VEGF/VEGFR family member is VEGF-A. In some embodiments, the VEGF/VEGFR family member is VEGF-C. In some embodiments, the VEGF/VEGFR family member is VEGF-D. In some embodiments, the VEGF/VEGFR family member is VEGFR-3.

In some embodiments, the pro-lymphatic formation factor targeted for inhibition is an integrin family member. In some embodiments, the integrin family member is selected from the group consisting of very late antigen-1 (VLA-1), integrin alpha 5 (ITGA5), and integrin alpha 9 (ITGA9). In some embodiments, the integrin family member is VLA-1. In some embodiments, the integrin family member is ITGA5. In some embodiments, the integrin family member is ITGA9.

In some embodiments, the pro-lymphatic formation factor targeted for inhibition is an angiopoietin family member. In some embodiments, the angiopoietin family member is selected from the group consisting of angiopoietin-1 (ANGPT1), angiopoietin-2 (ANGPT2 or Ang-2), and Tie2/TEK. In some embodiments, the angiopoietin family member is Ang-2.

In some embodiments, two or more inhibitors targeting two or more pro-lymphatic formation factors are administered to an eye in need thereof. In some embodiments the two or more inhibitors target two or more of VEGFR-3, VLA-1, ITGA5, ITGA9, and Ang-2. In some embodiments, the two or more pro-lymphatic formation factors targeted include VEGFR-3 and VLA-1. In some embodiments, the two or more pro-lymphatic formation factors targeted include VEGFR-3 and ITGA-5. In some embodiments, the two or more pro-lymphatic formation factors targeted include VEGFR-3 and ITGA-9. In some embodiments, the two or more pro-lymphatic formation factors targeted include VEGFR-3 and Ang-2. In some embodiments, the two or more pro-lymphatic formation factors targeted include VLA-1 and ITGA-5. In some embodiments, the two or more pro-lymphatic formation factors targeted include VLA-1 and ITGA-9. In some embodiments, the two or more pro-lymphatic formation factors targeted include VLA-1 and Ang-2. In some embodiments, the two or more pro-lymphatic formation factors targeted include ITGA-5 and ITGA-9. In some embodiments, the two or more pro-lymphatic formation factors targeted include ITGA-5 and Ang-2. In some embodiments, the two or more pro-lymphatic formation factors targeted include ITGA-9 and Ang-2.

Alternatively, in addition to the pro-lymphatic formation factors described above, a pro-lymphatic formation factor, or combination of pro-lymphatic formation factors, may be targeted for inhibition to inhibit or prevent lymphangiogenesis for the methods described above, including but not limited to, for example: a cytokine/chemokine, e.g., interleukin 8, interferon gamma, a member of CCR7/SLC axis, e.g., CCR7 and SLC; an extracellular matrix protein, e.g., CCBE1; a transcription factor, e.g., Sox18, Hhex; a guidance molecule, e.g., neuropilin 2 and SEMA7A; an FGF, e.g., FGF-2; a protein tyrosine phosphatase (PTP), e.g., PTPN14; a member of platelet factors, e.g., platelet-derived growth factor (PDGF-BB) or platelet factor 4; a lymphatic endothelial cell (LEC) polarity factor (e.g., Celsr1, Vangl2, Pdk2, and Fat4); a member of Notch family; or ARAF, SOS1, Apelin, Apelin, KIF11, REELIN, CALCRL, GJC2 (gap junction protein gamma-2), Rasip1, or FBXL7 (F-box and leucine-rich repeat protein 7).

Pro-lymphatic formation factor inhibitors suitable for administration to an eye in need thereof include, but are not limited to, for example (1) an agent capable of disrupting the pro-lymphatic formation factor's gene, including through genomic manipulation or knockout, thereby eliminating or reducing expression of the pro-lymphatic factor; (2) an siRNA capable of degrading the mRNA of a pro-lymphatic formation factor, thus reducing expression of the pro-lymphatic factor; (3) an antagonist antibody against the pro-lymphatic formation factor which inhibits or prevents the function of the pro-lymphatic formation factor; or (4) a small molecule capable of inhibiting or preventing the function of the pro-lymphatic formation factor. In some embodiments, the pro-lymphatic formation factor inhibitor is an siRNA. In some embodiments, the pro-lymphatic formation factor inhibitor is an antagonist antibody. Other strategies known in the art may also be used to disrupt the function or expression of a pro-lymphatic formation factor, or a combination of pro-lymphatic formation factors.

The inhibitor of a pro-lymphatic formation factor can be administered via any suitable route, systemically or, locally to an eye in need thereof. In some embodiments, the inhibitor of pro-lymphatic formation factor is administered to an eye in need thereof topically, via intracameral injection, via subconjunctival injection, via intravitreal injection, via suprachoroidal injection, via peribulbar injection, or via retrobulbar injection.

In some embodiments, the inhibitor of a pro-lymphatic formation factor is formulated as an eye drop, depot, bolus, inhibitor-loaded contact lens, suspension, solution, ophthalmic gel, or ointment.

In an alternative aspect, the inhibitor of lymphangiogenesis can be an agent that activates or stimulates an anti-lymphatic formation factor that inhibits lymphangiogenesis, as described further below. In some embodiments, the inhibitor of lymphangiogenesis is selected from the group consisting of: (1) an agent activating the expression of the anti-lymphatic formation factor; (2) an agonist antibody activating the anti-lymphatic formation factor; (3) microRNA or mimics inhibiting lymphatic formation, and (4) a small molecule activating the function of the anti-lymphatic formation factor. In some embodiments, the eye in need thereof is treated with a second therapy. In some embodiments, the second therapy is selected from the group consisting of an eye drop, an oral medication, a laser therapy, and a surgery. In some embodiments, the second therapy is selected from an eye drop that decreases the amount of fluid produced in the eye or improves the drainage of fluid from the eye, an oral medication that lowers IOP, a laser therapy (e.g., laser trabeculoplasty and laser iridotomy), and a surgery (e.g., implants, trabeculectomy). In some embodiments, the second therapy is a surgery. In some embodiments, the surgery is canaloplasty.

It is to be understood that both the foregoing general description and the following detailed description are exemplary and explanatory only and are not restrictive of the invention. Further, the accompanying drawings, which are incorporated in and constitute a part of this specification, illustrate embodiments of the invention and together with the description, serve to explain principles of the invention.

### BRIEF DESCRIPTION OF THE DRAWINGS

The drawings referenced herein form a part of the specification. Features shown in the drawing illustrate only some embodiments of the application, and not of all embodiments of the application, unless the detailed description explicitly indicates otherwise, and readers of the specification should not make implications to the contrary.

FIGS. 1A-C illustrate that the anti-VEGFR-3 treatment via siRNAs significantly lowered IOP and protected the eye from glaucomatous damage, as demonstrated in a mouse model of glaucoma. Intraocular hypertension was induced in normal eyes by laser photocoagulation of the episcleral veins. Anti-VEGFR-3 siRNA or control scrambled siRNA were administered locally via subconjunctival injection on Day 1 after laser.

FIG. 1A shows that compared to the control condition, IOP in the VEGFR-3 specific siRNA treated eyes was significantly reduced (P<0.05) after the treatment. IOP, measured in millimeters of mercury (mmHg), is represented on the y-axis for control (grey) and treatment (black) conditions over days following the laser photocoagulation on the x-axis.

FIG. 1B shows that VEGFR-3 specific siRNA treatment significantly reduced (*P<0.05) corneal edema, as measured in vivo by OCT. Central corneal thickness, measured in millimeters (mm), is represented on the y-axis in control (black) and treatment (grey) conditions on the x-axis.

FIG. 1C shows the summarized data with human Schlemm's canal cells showing that anti-VEGFR-3 siRNA treatment significantly inhibited Schlemm canal cell function, such as adhesion (*P<0.05). Fluorescence intensity measured in relative units, is represented on the y-axis in control (black) and treatment (grey) conditions on the x-axis.

FIGS. 2A-D show that the anti-VLA-1 antibody treatment significantly lowered IOP and protected the ocular tissues from glaucomatous damage, as demonstrated in a mouse model of glaucoma. Intraocular hypertension was induced in the right eyes of normal eyes by laser. Anti-VLA-1 antibody or control were administered locally via subconjunctival injection and started on Day 1 after laser.

FIG. 2A shows that compared to the control condition, IOP in the anti-VLA-1 antibody treated eyes was significantly reduced (P<0.05). IOP, measured in millimeters of mercury (mmHg), is represented on the y-axis for control (grey) and treatment (black) conditions over days following the laser photocoagulation on the x-axis.

FIG. 2B-D show the summarized data showing anti-VLA-1 antibody treatment protected the cornea from edema (FIG. 2B) and reduced RNFL (retinal nerve fiber layer) thinning (FIG. 2C) and retinal ganglion cell (RGC) death (FIG. 2D) as well. Central corneal thickness and RNFL thickness were measured in vivo by OCT. *P<0.05. n.s. not significant.

FIG. 2B shows that anti-VLA-1 antibody treatment significantly reduced (*P<0.05) corneal edema, as measured in vivo by OCT. Central corneal thickness, measured in micrometers (μm), is represented on the y-axis in control (grey) and treatment (black) conditions on the x-axis.

FIG. 2C shows that anti-VLA-1 antibody treatment significantly reduced (*P<0.05) RNFL thinning, as measured in vivo by OCT. RNFL thickness, measured in micrometers (μm), is represented on the y-axis in control (grey) and treatment (black) conditions on the x-axis.

FIG. 2D shows that anti-VLA-1 antibody treatment significantly reduced (*P<0.05) RGC death as compared to the control, non-treated eye. Relative RGC number, measured in percent (%), is represented on the y-axis for the control and lasered eye on the x-axis in control mice (grey) and treated mice (black). There was no significant difference (n.s., not significant) in the relative number of RGCs between control mice and VLA-1 antibody treated mice in the eyes which were not administered laser photocoagulation. In the eyes which were induced to hypertension by laser administration, the anti-VLA-1 antibody treated mice had significantly more (*P<0.05) RGCs.

FIG. 3A shows that the anti-VLA-1 treatment via siRNAs significantly lowered (*P<0.05) IOP, as demonstrated in the mouse model of glaucoma. Intraocular hypertension was induced in normal eyes by laser. Anti-VLA-1 siRNA or control scrambled siRNA were administered locally via subconjunctival injection on Day 1 after laser. IOP, measured in millimeters of mercury (mmHg) on Day 3 after laser, is represented on the y-axis for control (white) and treatment (grey) conditions on the x-axis. IOP in the anti-VLA-1 siRNA treated eyes was significantly reduced (*P<0.05) compared to the control condition.

FIG. 3B and FIG. 3C show the summarized data with human Schlemm's canal cells showing that anti-VLA-1 siRNA treatment inhibited Schlemm canal cell functions, such as adhesion (FIG. 3B) and tube formation (FIG. 3C) (*P<0.05).

FIG. 3B shows the summarized data with human Schlemm's canal cells demonstrating that anti-VLA-1 siRNA treatment significantly inhibited Schlemm canal cell adhesion (*P<0.05). Fluorescence intensity is represented in relative units on the y-axis in control (white) and treatment (grey) conditions on the x-axis.

FIG. 3C shows that anti-VLA-1 siRNA treatment significantly inhibited (*P<0.05) Schlemm canal cell tube formation. The number of meshes is represented in relative percent (%) on the y-axis for control (white) and treatment (grey) conditions on the x-axis. Significantly fewer (*P<0.05) meshes were observed in the anti-VLA-1 siRNA treated condition compared with the control condition.

FIGS. 4A-C show that anti-Ang-2 treatment via siRNAs significantly lowered IOP, as demonstrated in the mouse model of glaucoma. Intraocular hypertension was induced in normal eyes by laser. Anti-Ang-2 siRNA or control scrambled siRNA were administered locally via subconjunctival injection on Day 1 after laser.

FIG. 4A shows that compared to the control condition, IOP in the anti-Ang-2 siRNA treated eyes was significantly reduced (P<0.05). IOP, measured in millimeters of mercury (mmHg), is represented on the y-axis for control (grey) and treatment (black) conditions over days following the laser photocoagulation on the x-axis.

(FIG. 4B and FIG. 4C) Summarized data with human Schlemm's canal cells showing that anti-Ang-2 siRNA treatment inhibited Schlemm canal cell functions, such as adhesion (FIG. 4B) and tube formation (FIG. 4C) (*P<0.05).

FIG. 4B shows the summarized data with human Schlemm's canal cells demonstrating that anti-Ang-2 siRNA treatment significantly inhibited Schlemm canal cell adhesion (*P<0.05). Fluorescence intensity is represented in relative units on the y-axis in control (white) and treatment (grey) conditions on the x-axis.

FIG. 4C shows that anti-Ang-2 siRNA treatment significantly inhibited (*P<0.05) Schlemm canal cell tube formation. The number of meshes is represented in relative percent (%) on the y-axis for control (white) and treatment (grey) conditions on the x-axis. Significantly fewer (*P<0.05) meshes were observed in the anti-Ang-2 siRNA treated condition compared with the control condition.

FIG. 5A shows that the anti-ITGA5 treatment via siRNAs significantly lowered IOP (*P<0.05), as demonstrated in the mouse model of glaucoma. Intraocular hypertension was induced in normal eyes by laser. Anti-ITGA5 siRNA or control scrambled siRNA were administered locally via subconjunctival injection on Day 1 after laser. IOP, measured in millimeters of mercury (mmHg) on Day 3 after laser, is represented on the y-axis for control (white) and treatment (grey) conditions over days following the laser photocoagulation on the x-axis. IOP in the anti-ITGA5 siRNA treated eyes was significantly reduced (*P<0.05) compared to the control condition.

FIG. 5B shows the summarized data with human Schlemm's canal cells showing that anti-ITGA5 siRNA treatment inhibited Schlemm canal cell function, such as adhesion (*P<0.05). Fluorescence intensity is represented in relative units on the y-axis in control (white) and treatment (grey) conditions on the x-axis.

### DETAILED DESCRIPTION OF THE INVENTION

The following detailed description of exemplary embodiments of the application refers to the accompanying drawings that form a part of the description. The drawings illustrate specific exemplary embodiments in which the application may be practiced. The detailed description, including the drawings, describes these embodiments in sufficient detail to enable those skilled in the art to practice the application. Those skilled in the art may further utilize other embodiments of the application, and make logical, mechanical, and other changes without departing from the spirit or scope of the application. Readers of the following detailed description should, therefore, not interpret the description in a limiting sense, and only the appended claims define the scope of the embodiment of the application.

In this application, the use of the singular includes the plural unless specifically stated otherwise. In this application, the use of “or” means “and/or” unless stated otherwise. Furthermore, the use of the term “including” as well as other forms such as “includes” and “included” is not limiting. Additionally, the section headings used herein are for organizational purposes only and are not to be construed as limiting the subject matter described.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, some specifically embodied methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, the term “about” means approximately or nearly and in the context of a numerical value or range set forth herein ±10% of the numerical value or range recited or claimed.

As used herein, the term “administering” means providing a pharmaceutical agent or composition to a subject, and includes, but is not limited to, administering by a medical professional and self-administering.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)2, Fab, Fv, and rIgG). See also, e.g., Pierce Catalog and Handbook, 1994-1995 (Pierce Chemical Co., Rockford, Ill.); Kuby, J., Immunology, 3^{rd} Ed., W.H. Freeman & Co., New York (1998). The term antibody also includes bivalent or bispecific molecules, diabodies, triabodies, and tetrabodies. The term “antibody” further includes both polyclonal and monoclonal antibodies.

It is noted that in this disclosure, terms such as “comprises”, “comprised”, “comprising”, “contains”, “containing” and the like have the meaning attributed in United States patent law; they are inclusive or open-ended and do not exclude additional, un-recited elements or method steps. Terms such as “consisting essentially of” and “consists essentially of” have the meaning attributed in United States patent law; they allow for the inclusion of additional ingredients or steps that do not materially affect the basic and novel characteristics of the claimed invention. The terms “consists of” and “consisting of” have the meaning ascribed to them in United States patent law; namely that these terms are close ended.

As used herein, the term “inhibitor” refers to biological or chemical substance that interferes with or otherwise reduces the physiological and/or biochemical action of another biological or chemical molecule. In some embodiments, the inhibitor specifically binds to the other molecule. For example, expression of a pro-lymphatic factor is suppressed by at least about 5%, 10%, 15%, 20%, 25%, 30%, 35%, 40%, 45%, or 50% by administration of the inhibitor described in the present invention. In some embodiments, a pro-lymphatic factor is suppressed by at least about 60%, 70%, or 80% by administration of an inhibitor described in the present invention. In some embodiments, a pro-lymphatic factor is suppressed by at least about 85%, 90%, or 95% by administration of an inhibitor described in the present invention.

As used herein, the term “small interfering RNA (siRNA)” (also known as short interfering RNA or silencing RNA) refers to a class of double-stranded RNA and/or non-coding RNA molecules. The siRNA described in the present application may be used for silencing protein-coding genes. The siRNA described in the present application may be used to target mRNA of a specific gene to produce a gene silencing effect. For example, the siRNA may interfere with the expression of specific genes with complementary nucleotide sequences by degrading mRNA after transcription, and preventing translation. The siRNA includes two RNA strands that are sufficiently complementary to hybridize to form a duplex structure under suitable conditions, with one siRNA strand (the antisense strand) including a region of complementarity that is substantially complementary to a target sequence, the other siRNA strand (the sense strand) including a region that is complementary to the antisense strand. In some embodiments, the duplex siRNA structure is between 15 and 30 or between 25 and 30, or between 18 and 25, or between 19 and 24, or between 19 and 21, or 19, 20, or 21 base pairs in length. In some embodiments, the siRNA duplex is 19 base pairs in length. In some embodiments, the siRNA duplex is 21 base pairs in length. Each strand of the siRNA duplex can be the same length or of different lengths. The siRNAs as described in the present invention can include one or more single-stranded overhang(s) of one or more nucleotides. In some embodiments, at least one end of the dsRNA has a single-stranded nucleotide overhang of 1 to 4, generally 1 or 2 nucleotides. In some embodiments, the siRNA is chemically modified to enhance stability. The nucleic acid molecules described in the invention may be synthesized and/or modified by methods established in the art, for example those described in “Current protocols in nucleic acid chemistry,” Beaucage, S. L. et al. (Eds.), John Wiley & Sons, Inc., New York, NY, USA, which is hereby incorporated herein by reference.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

As used herein, the term “antisense strand” refers to the strand of an siRNA, which includes a sequence region that is substantially complementary to a target sequence.

As used herein, the term “sense strand,” refers to the strand of an siRNA, that includes a region that is substantially complementary to a region of the antisense strand.

As used herein, the terms “subject”, “host”, and “patient” are used interchangeably. As used herein, the “patient,” “host,” or “subject” treated is typically a human patient, although it is to be understood the methods described herein are effective with respect to other animals, such as mammals. In particular embodiments, the patient, host, or subject is a human.

As used herein, the term “therapeutically effective amount” means the amount of agent that is sufficient to prevent, treat, reduce and/or ameliorate the symptoms and/or underlying causes of any disorder or disease, or the amount of an agent sufficient to produce a desired effect on a cell.

The term “treatment,” “treat,” or “treating” refers to a method of reducing the damages or severity of a disease or symptom of a disease. Thus, in the disclosed method, treatment can refer to a 10%, 20%, 30%, 40%, 50%, 60%, 70%, 80%, 90%, or 100% reduction in the damage or severity of disease or symptom of the disease. For example, a method of treating a disease is considered to be a treatment if there is a 10% reduction in one or more symptoms of the disease in as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Lymphangiogenesis and Glaucoma

The term “lymphangiogenesis” as used herein refers to the process of forming new lymphatic vessels from pre-existing vessels or lymphatic endothelial precursor cells. The lymphatic system plays an important role in maintaining fluid balance and immune function in the body. Lymphatic vessels are responsible for transporting lymphatic fluid, which contains immune cells and other important components, throughout the body.

Lymphangiogenesis occurs during normal development, wound healing, and certain pathological conditions, such as cancer and inflammation. The process involves multiple facets, such as the activation of lymphatic endothelial cells, the patterning and maintenance of vessels, and the expression of growth factors, such as vascular endothelial growth factors and receptors (e.g. VEGF-C, VEGF-D, VEGFR-3) and angiopoietin factors, which promote the formation, patterning, and maintenance of lymphatic vessels. The lymphatic network penetrates many tissues in the body, and its dysfunction has been found in a broad spectrum of disorders, such as cancer metastasis, inflammatory and immune diseases, tissue and organ (heart and kidney) transplant rejection, obesity, hypertension, and lymphedema (Chen L. Ocular lymphatics: state-of-the-art review. Lymphology. 42:66-76 (2009)).

Some studies suggest that lymphangiogenesis may play a role in the pathogenesis of glaucoma. For example, studies have suggested that lymphatic vessels may be involved in the drainage of aqueous humor, which is important in maintaining normal eye pressure. Researchers have found that disruption of lymphatic vessels may contribute to elevated intraocular pressure (IOP) (Aspelund, A. et al. The Schlemm's canal is a VEGF-C/VEGFR-3-responsive lymphatic-like vessel. J Clin Invest. 124 (9): 3975-3986 (2014 September); Thomson, B. R. et al. A lymphatic defect causes ocular hypertension and glaucoma in mice. J Clin Invest. 124 (10): 4320-4324 (2014 October; Epub 2014 Sep. 9); Kim, J. et al. Impaired angiopoietin/Tie2 signaling compromises Schlemm's canal integrity and induces glaucoma. J Clin Invest. 127 (10): 3877-3896 (2017 Oct. 2; Epub 2017 Sep. 18)), which is a key risk factor for glaucoma. It has been proposed that inducing or activating lymphangiogenesis may potentially treat glaucoma (see, e.g., WO2015110701A1, incorporated herein by reference).

The present disclosure is based on the surprising discovery that inhibiting a pro-lymphatic factor lowers IOP and protects the eye from glaucomatous damage. Therefore, in one aspect, the present disclosure provides a method for preventing or treating glaucoma. In one embodiment, the method comprises administering to an eye in need thereof a therapeutically effective amount of an inhibitor of a pro-lymphatic factor, thereby lowering IOP of the eye. In some embodiments, the present disclosure provides a method for preventing or treating primary glaucoma. In some embodiments, the primary glaucoma is primary open-angle glaucoma (POAG). In some embodiments, the primary glaucoma is angle-closure glaucoma. In some embodiments, the primary glaucoma is congenital glaucoma. In some embodiments, the primary glaucoma is normal tension glaucoma.

In some embodiments, the present disclosure provides a method for preventing or treating an IOP-mediated ocular disorder. In some embodiments, the IOP-mediated ocular disorder is selected from the group consisting of a secondary glaucoma, glaucomatous optic neuropathy (GON), ocular hypertension, open angle glaucoma, angle-closure glaucoma, and congenital glaucoma.

### Lymphangiogenesis and the Schlemm's Canal

The Schlemm's canal is a critical structure in the regulation of aqueous humor drainage and IOP as a crucial component of the conventional outflow pathway. The Schlemm's canal itself accounts for 70-90% of the total aqueous humor outflow in humans. The endothelial cells that line the Schlemm's canal are a primary site of resistance to aqueous humor drainage and are major determinants of overall IOP. When Schlemm's canal resistance increases in response to some pathological insult, elevated IOP is observed. The Schlemm's canal therefore may play a key role in the etiology of glaucoma.

It was previously demonstrated that several lymphatic markers, for example PROX-1 and VEGF-C, were involved in the development of the Schlemm's canal (Aspelund, A. et al. The Schlemm's canal is a VEGF-C/VEGFR-3-responsive lymphatic-like vessel. J Clin Invest. 124 (9): 3975-3986 (2014 September)), suggesting the Schlemm's canal may exhibit a lymphatic-like phenotype. While glaucoma has been associated with dysregulated lymphatic systems, other studies have concluded that there is a lack of similarity between lymphatics and aqueous drainage canals (Chen, L. Ocular lymphatics: state-of-the-art review. Lymphology. 42 (2): 66-76 (2009 June).

### Pro-Lymphatic Formation Factors

In some aspects of the present invention, a lymphatic formation factor is targeted to reduce, inhibit, or prevent ocular lymphangiogenesis. As used herein, a lymphatic formation factor refers to a gene, RNA or protein that is involved in the formation or maintenance of lymphatic vessels. The lymphatic formation factor may induce (pro-lymphatic) or inhibit (anti-lymphatic) lymphangiogenesis. Therefore, in some embodiments, the inhibitor of a pro-lymphatic factor inhibits a pro-lymphatic formation factor, wherein the pro-lymphatic formation factor induces or maintains ocular lymphangiogenesis. In some embodiments, the inhibitor of ocular lymphangiogenesis activates an anti-lymphatic formation factor, wherein the anti-lymphatic formation factor inhibits ocular lymphangiogenesis.

In some embodiments, the pro-lymphatic formation factor disclosed herein is a member of VEGF/VEGFR family. Examples of VEGF/VEGFR family member that is a pro-lymphatic formation factor include VEGF-A, VEGF-B, VEGF-C, VEGF-D, VEGF-E, VEGFR-1, VEGFR-2, and VEGFR-3. In some embodiments, the pro-lymphatic formation factor is selected from the group consisting of VEGF-C, VEGF-D, and VEGFR-3. In some embodiments, the pro-lymphatic formation factor is VEGFR-3.

In some embodiments, the pro-lymphatic formation factor disclosed herein is an integrin. Examples of an integrin that is a pro-lymphatic formation factor include VLA-1, integrin alpha 5 (ITGA5), and integrin alpha 9 (ITGA9). In some embodiments, the pro-lymphatic formation factor is VLA-1. In some embodiments, the pro-lymphatic formation factor is ITGA5. In some embodiments, the pro-lymphatic formation factor is ITGA9.

In some embodiments, the pro-lymphatic formation factor disclosed herein is an angiopoietin. Examples of angiopoietin that is a pro-lymphatic formation factor include angiopoietin 1 (ANGPT1), angiopoietin-2 (ANGPT2 or Ang-2), and Tie2/TEK. In some embodiments, the pro-lymphatic formation factor is Ang-2.

In some embodiments, the pro-lymphatic formation factor disclosed herein is a cytokine/chemokine. Examples of cytokine/chemokine that is a pro-lymphatic formation factor include interleukin 8, interferon gamma, CCR7, and SLC.

In some embodiments, the pro-lymphatic formation factor disclosed herein is an extracellular matrix protein. Examples of extracellular matrix protein that is a pro-lymphatic formation factor include CCBE1.

In some embodiments, the pro-lymphatic formation factor disclosed herein is a transcription factor. Examples of transcription factor that is a pro-lymphatic formation factor include Sox18 and Hhex.

In some embodiments, the pro-lymphatic formation factor disclosed herein is a guidance molecule. Examples of guidance molecule that is a pro-lymphatic formation factor include neuropilin 2 and SEMA7A.

In some embodiments, the pro-lymphatic formation factor disclosed herein is an FGF. Examples of FGF that is a pro-lymphatic formation factor include FGF-2.

In some embodiments, the pro-lymphatic formation factor disclosed herein is a protein tyrosine phosphatase (PTP). Examples of PTP that is a pro-lymphatic formation factor include PTPN14.

In some embodiments, the pro-lymphatic formation factor disclosed herein is a platelet factor. Examples of platelet factors that is a pro-lymphatic formation factor include platelet factor 4 or platelet-derived growth factor (e.g., PDGF-BB).

In some embodiments, the pro-lymphatic formation factor disclosed herein is LEC polarity factor (e.g., Celsr1, Vangl2, Pdk2, and Fat4). Examples of LEC polarity factors that is a pro-lymphatic formation factor include Celsr1, Vangl2, Pdk2, and Fat4.

In some embodiments, the pro-lymphatic formation factor disclosed herein is a member of Notch family.

In some embodiments, the pro-lymphatic formation factor disclosed herein is ARAF, SOS1, Apelin, KIF11, REELIN, CALCRL, GJC2 (gap junction protein gamma-2), Rasip1, or FBXL7 (F-box and leucine-rich repeat protein 7).

In some embodiments, the pro-lymphatic formation factor is a major pro-lymphatic formation factor. In some embodiments, the major pro-lymphatic formation factor is selected from the group consisting of VEGFR-3, VEGF-C, VEGF-D, VEGF-A, VLA-1, angiopoietin-2, Tie-2, and integrin alpha 5.

In some embodiments, the pro-lymphatic formation factor is a minor pro-lymphatic formation factor. In some embodiments, the minor pro-lymphatic formation factor is selected from the group consisting of VEGF-B, VEGF-E, PIGF, VEGFR-1, VEGFR-2, ANGPT1, ITGA9, TEK, interleukin 8, interferon gamma, CCR7, SLC, CCBE1, Sox18, Hhex, neuropilin 2 and SEMA7AFGF-2, PTPN14, PDGF-BB, platelet factor 4, Celsr1, Vangl2, Pdk2, Fat4, a Notch family member, ARAF, SOS1, Apelin, KIF11, REELIN, CALCRL, GJC2, Rasip1, and FBXL7.

### VEGF/VEGFR Family

The vascular endothelial growth factor (VEGF) family regulates blood and lymphatic vessel processes. In some embodiments, the pro-lymphatic formation factor to be inhibited as disclosed herein is a member of VEGF/VEGFR family. Examples of VEGF/VEGFR family member that is a pro-lymphatic formation factor include VEGF-A, VEGF-B, VEGF-C, VEGF-D, VEGF-E, VEGFR-1, VEGFR-2, and VEGFR-3. In some embodiments, the pro-lymphatic formation factor is selected from the group consisting of VEGF-C, VEGF-D, and VEGFR-3.

### Vascular Endothelial Growth Factor Receptor 3 (VEGFR-3)

In some embodiments, the pro-lymphatic formation factor to be inhibited as disclosed herein is VEGFR-3. VEGFR-3 belongs to the VEGF family. Previous studies have shown that VEGFR-3 mediates lymphangiogenesis in the cornea and other tissues, and its inhibition suppresses transplant rejection, tumor growth, and metastasis (Yuen, D. et al. Combined blockade of VEGFR-2 and VEGFR-3 inhibits inflammatory lymphangiogenesis in early and middle stages. Invest Ophthalmol Vis Sci. 52 (5): 2593-2597 (2011 Apr. 20)). The potential role of VEGFR-3 in primary glaucoma, and the specificity thereof, however, has not been determined previously.

### Integrin Family

In some embodiments, the pro-lymphatic formation factor to be inhibited as disclosed herein is an integrin, such as beta 1 integrin. Integrins are heterodimeric transmembrane receptors which link the actin cytoskeleton to the ECM to influence gene expression (Vigneault, F. et al. Control of integrin genes expression in the eye. Progress in Retinal and Eye Res. 26:99-161 (2007)). Examples of beta 1 integrin that is a pro-lymphatic formation factor include VLA-1, integrin alpha 5 (ITGA5), and integrin alpha 9 (ITGA9). In some embodiments, the pro-lymphatic formation factor is VLA-1. In some embodiments, the pro-lymphatic formation factor is ITGA5. In some embodiments, the pro-lymphatic formation factor is ITGA9.

### Integrin Alpha 5 (ITGA5)

In some embodiments, the pro-lymphatic formation factor to be inhibited as disclosed herein is ITGA5. Integrin alpha 5 (ITGA5) belongs to the integrin alpha chain family that mediates cell surface adhesion and signaling. ITGA5 is a preprotein which is proteolytically cleaved to produce light and heavy chains that comprise the alpha 5 subunit. Previously, it was reported that ITGA-5 mediates corneal inflammatory lymphangiogenesis, which is suppressed by ITGA-5 blockade (Dietrich T, et al. Inhibition of Inflammatory Lymphangiogenesis by Integrin 5 Blockade. Am J Pathol. 2007 July; 171 (1): 361-72).

### Very Late Antigen 1 (VLA-1)

In some embodiments, the pro-lymphatic formation factor to be inhibited as disclosed herein is VLA-1. VLA-1 (very late antigen-1), also known as integrin α1β1, is a receptor for collagen and laminin. VLA-1 is expressed on lymphatic endothelial cells (LECs). Previously, it was reported that VLA-1 mediates corneal inflammatory lymphangiogenesis, which is suppressed by VLA-1 blockade (Grimaldo, S. et al. Very late antigen-1 mediates corneal lymphangiogenesis. Invest Ophthalmol Vis Sci. 52 (7): 4808-4812 (2011 Jul. 1)). It was also reported that corneal transplantation-associated lymphangiogenesis is suppressed in VLA-1 knockout mice (Chen, L. et al. Very late antigen 1 blockade markedly promotes survival of corneal allografts. Arch Ophthalmol. 125:783-788 (2007)). The molecular and cellular mechanisms of VLA-1 in other ocular diseases such as glaucoma, however, have not been previously determined.

### Angiopoietin Family

In some embodiments, the pro-lymphatic formation factor to be inhibited as disclosed herein is an angiopoietin. The angiopoietin (ANGPT) family is a signaling pathway comprising ligands (e.g., angiopoietin 1 (ANGPT1), angiopoietin 2 (ANGPT2 or Ang-2)) which activate Tie2/TEK. Examples of angiopoietin that is a lymphatic formation factor include angiopoietin 1 (ANGPT1), angiopoietin-2 (ANGPT2 or Ang-2), and Tie2/TEK. In some embodiments, the pro-lymphatic formation factor is Ang-2.

### Angiopoietin-2 (Ang-2) (ANGPT2)

In some embodiments, the pro-lymphatic formation factor to be inhibited as disclosed herein is Ang-2. Angiopoietin-2 (Ang-2 or ANGPT2) belongs to the angiopoietin-Tie family. The particular function of Ang-2 in the lymphatic system is yet to be fully understood. Previously, it was reported that Ang-2 deficiency leads to lymphatic defects in development and inflammation (Dellinger, M. et al. Defective remodeling and maturation of the lymphatic vasculature in angiopoietin-2 deficient mice. Dev Biol. 319:309-320 (2008); Yuen, D. et al. Role of angiopoietin-2 in corneal lymphangiogenesis. Invest Ophthalmol Vis Sci. 55:3320-3327 (2014); Zheng, W. et al. Angiopoietin 2 regulates the transformation and integrity of lymphatic endothelial cell junctions. Genes Dev. 28:1592-1603 (2014)). The role of Ang-2 in glaucoma, however, has not been fully elucidated.

### Cytokines/Chemokines

In some embodiments, the pro-lymphatic formation factor to be inhibited as disclosed herein is a cytokine/chemokine. Cytokines or chemokines are implicated in lymphangiogenesis (Sainz-Jaspeado, M. & Claesson-Welsh, L. Cytokines regulating lymphangiogenesis. Curr Opin in Immunol. 53:58-63 (2018)). Chemokines are small protein cytokines that act as chemoattractants which are also shown to be involved in lymphatic systems (Farnsworth, R. H. et al. The Interplay Between Lymphatic Vessels and Chemokines. Front Immunol. 10:518 (2019 Apr. 12)). Examples of cytokine/chemokine that is a lymphatic formation factor include interleukin 8, interferon gamma, CCR7, and SLC.

### Extracellular Matrix Proteins

In some embodiments, the pro-lymphatic formation factor to be inhibited as disclosed herein is an extracellular matrix protein. Examples of extracellular matrix protein that is a lymphatic formation factor include CCBE1. CCBE1 binds the ECM and contributes to VEGF-C-mediated activation of VEGFR-3 (Brouillard, P. et al. Genetics of lymphatic anomalies. J Clin Invest. 124 (3): 898-904 (2014 March; Epub 2014 Mar. 3).

### Transcription Factors

In some embodiments, the pro-lymphatic formation factor to be inhibited as disclosed herein is a transcription factor. Examples of transcription factor that is a lymphatic formation factor include Sox18 and Hhex. Several transcription factors act upstream or downstream of VEGFR-3 (Brouillard, P. et al. Genetics of lymphatic anomalies. J Clin Invest. 124 (3): 898-904 (2014 March; Epub 2014 Mar. 3).

### Guidance Molecules

In some embodiments, the pro-lymphatic formation factor to be inhibited as disclosed herein is a guidance molecule. Examples of guidance molecule that is a lymphatic formation factor include neuropilin 2 and SEMA7A.

### Fibroblast Growth Factor (FGF) Family

In some embodiments, the pro-lymphatic formation factor to be inhibited as disclosed herein is a member of the fibroblast growth factor (FGF) family. FGFs are mitogens that regulate a variety of biological processes including cellular proliferation, differentiation, and survival, and are involved in several diseases (Xie, Y. et al. FGF/FGFR signaling in health and disease. Signal Transduct Target Ther. 5 (1): 181 (2020 Sep. 2)). Examples of FGF that is a lymphatic formation factor include FGF-2.

### Protein Tyrosine Phosphatase (PTP)

In some embodiments, the pro-lymphatic formation factor to be inhibited as disclosed herein is a protein tyrosine phosphatase (PTP). In some embodiments, the lymphatic formation factor is PTPN14. PTPN14 is a phosphatase that interacts with VEGF-C-activated VEGFR3 (Oliver, G. et al. The Lymphatic Vasculature in the 21st Century: Novel Functional Roles in Homeostasis and Disease. Cell. 182 (2): 270-296 (2020 Jul. 23)).

### Platelet Factors

In some embodiments, the pro-lymphatic formation factor to be inhibited as disclosed herein is a platelet factor. Examples of platelet factors that is a lymphatic formation factor include platelet factor 4 or platelet-derived growth factor (e.g., PDGF-BB). Platelet factors are associated with the lymphatic vasculature (Ma, W. et al. Platelet factor 4 is a biomarker for lymphatic-promoted disorders. JCI Insight. 5 (13): e135109 (2020 Jul. 9)). In some embodiments, the lymphatic formation factor is platelet factor 4 (PF4). In some embodiments, the lymphatic formation factor is platelet-derived growth factor (PDGF-BB).

### Lymphatic Endothelial Cell (LEC) Polarity Factor

In some embodiments, the pro-lymphatic formation factor to be inhibited as disclosed herein is a LEC polarity factor. Lymphatic endothelial cell (LEC) polarity factors control planar cell polarity and organization of lymphathic vessels. For example, Fat4 acts as a cell polarity regulator that is required for lymphatic vasculature morphogenesis during development (Betterman, K. L. et al. Atypical cadherin FAT4 orchestrates lymphatic endothelial cell polarity in response to flow. J Clin Invest. 130 (6): 3315-3328 (2020 Jun. 1)). Examples of LEC polarity factors that is a lymphatic formation factor include Celsr1, Vangl2, Pdk2, and Fat4.

### Notch Family

In some embodiments, the pro-lymphatic formation factor disclosed herein is a member of Notch family. The notch pathway of receptors (Notch1-4) and ligands (Dll1/3/4 and Jagged1/2) induce intercellular signaling to regulate cellular development, proliferation, and differentiation. The Notch pathway has also been implicated in lymphangiogenesis and lymphatic differentiation (Niessen, K. et al. The Notch1-Dll4 signaling pathway regulates mouse postnatal lymphatic development. Blood. 118 (7): 1989-1997 (2011 Aug. 18)).

### Inhibitor of a Pro-Lymphatic Formation Factor

In some embodiments, the inhibitor of a pro-lymphatic factor is selected from the group consisting of a polynucleotide, a protein, a polypeptide, and a gene editing composition. The inhibitor of a pro-lymphatic factor can be a nucleotide (e.g., an siRNA), a protein (e.g., an antibody, a recombinant protein), a peptide, a small molecule, or a gene editing composition (e.g., CRISPR/Cas, gRNA). In some embodiments, the inhibitor of a pro-lymphatic factor is selected from the group consisting of a gene-disrupting agent, an siRNA, an antagonist antibody, a recombinant protein, and a small molecule.

In some embodiments, the inhibitor of a pro-lymphatic factor is an agent disrupting the gene of the pro-lymphatic formation factor. In some embodiments, the inhibitor of a pro-lymphatic factor is an siRNA against the pro-lymphatic formation factor. In some embodiments, the inhibitor of a pro-lymphatic factor is an antagonist antibody or a recombinant protein against the pro-lymphatic formation factor. In some embodiments, the inhibitor of a pro-lymphatic factor is a small molecule inhibiting the function of the pro-lymphatic formation factor.

In some embodiments, the inhibitor of a pro-lymphatic factor is an agent that disrupts the gene of the pro-lymphatic formation factor. In some embodiments, the gene disrupting agent comprises a gene editing composition. In some embodiments, the gene editing composition comprises CRISPR/Cas9 and gRNA. The gene editing compositions described in the invention may be generated and used according to methods established in the art, for example those described in “Genome engineering using the CRISPR-Cas9 system,” Ran, F. A. et al. Nature Protocols 11 (8) (2013), which is hereby incorporated herein by reference.

In some embodiments, the lymphangiogenesis inhibitor is an agent that activates the expression of an anti-lymphatic formation factor. In some embodiments, the lymphangiogenesis inhibitor is an agonist antibody against an anti-lymphatic formation factor. In some embodiments, the lymphangiogenesis inhibitor is a small molecule that activates the function of an anti-lymphatic formation factor.

Exemplary siRNA molecules for inhibiting certain pro-lymphatic formation factors are included in Table 1.

TABLE 1
Exemplary siRNA as Inhibitors of Pro-lymphatic Factors

| | Cat | Assay | | |
|---|---|---|---|---|
| Name | number | ID | Sequence (“→”) sense | Sequence (“→”) antisense |
| VEGFR- | 4457308 | s5294 | CCAGCAUCCUGACCAU | UGGAUGGUCAGGAUGCU |
| 3 | | | CCAtt (SEQ ID NO: 1) | GGag (SEQ ID NO: 2) |
| VEGFR- | AM16704 | 157561 | GCUGGUUUUGAACUGU | UGUACAGUUCAAAACCA |
| 3 | | | ACAtt (SEQ ID NO: 3) | GCtt (SEQ ID NO: 4) |
| VEGFR- | 4457308 | 157561 | GCUGGUUUUGAACUGU | UGUACAGUUCAAAACCA |
| 3 | | | ACAtt (SEQ ID NO: 3) | GCtt (SEQ ID NO: 4) |
| VEGFR- | 4392420 | s5294 | CCAGCAUCCUGACCAU | UGGAUGGUCAGGAUGCU |
| 3 | | | CCAtt (SEQ ID NO: 1) | GGag (SEQ ID NO: 2) |
| VLA-1 | 4390824 | s7532 | GGACUUUAAUCUUACC | AUCGGUAAGAUUAAAGU |
| | | | GAUtt (SEQ ID NO: 5) | CCaa (SEQ ID NO: 6) |
| VLA-1 | 4457308 | s7532 | GGACUUUAAUCUUACC | AUCGGUAAGAUUAAAGU |
| | | | GAUtt (SEQ ID NO: 5) | CCaa (SEQ ID NO: 6) |
| VLA-1 | AM16704 | 500900 | GAGUGAAAUUCAUUG | UAUCCAAUGAAUUUCAC |
| | | | GAUAtt (SEQ ID NO: 7) | UCct (SEQ ID NO: 8) |
| VLA-1 | 4457308 | 500900 | GAGUGAAAUUCAUUG | UAUCCAAUGAAUUUCAC |
| | | | GAUAtt (SEQ ID NO: 7) | UCct (SEQ ID NO: 8) |
| VLA-1 | 4390771 | s99598 | GGAUCAACUUUAGUCA | UGGUGACUAAAGUUGAU |
| | | | CCAtt (SEQ ID NO: 9) | CCaa (SEQ ID NO: 10) |
| VLA-1 | 4457308 | s99598 | GGAUCAACUUUAGUCA | UGGUGACUAAAGUUGAU |
| | | | CCAtt (SEQ ID NO: 9) | CCaa (SEQ ID NO: 10) |
| Ang-2 | 4392420 | s1359 | CCUUCCAACUUGAACG | UUCCGUUCAAGUUGGAA |
| | | | GAAtt (SEQ ID NO: 11) | GGac (SEQ ID NO: 12) |
| Ang-2 | 4457308 | s1359 | CCUUCCAACUUGAACG | UUCCGUUCAAGUUGGAA |
| | | | GAAtt (SEQ ID NO: 11) | GGac (SEQ ID NO: 12) |
| Ang-2 | 4390817 | S62324 | CCCUUAUGGACGAAAU | UAAAUUUCGUCCAUAAG |
| | | | UUAtt (SEQ ID NO: 13) | GGct (SEQ ID NO: 14) |
| Ang-2 | 4457308 | S62324 | CCCUUAUGGACGAAAU | UAAAUUUCGUCCAUAAG |
| | | | UUAtt (SEQ ID NO: 13) | GGct (SEQ ID NO: 14) |
| ITGA5 | 4390824 | s7549 | GCAGAGAGAUGAAGA | UAGAUCUUCAUCUCUCU |
| | | | UCUAtt (SEQ ID NO: 15) | GCaa (SEQ ID NO: 16) |
| ITGA5 | 4457308 | s7549 | GCAGAGAGAUGAAGA | UAGAUCUUCAUCUCUCU |
| | | | UCUAtt (SEQ ID NO: 15) | GCaa (SEQ ID NO: 16) |
| ITGA5 | 4390817 | s68428 | CCUCAGGAAUGAAUCA | UUCUGAUUCAUUCCUGA |
| | | | GAAtt (SEQ ID NO: 17) | GGta (SEQ ID NO: 18) |
| ITGA5 | 4457308 | s68428 | CCUCAGGAAUGAAUCA | UUCUGAUUCAUUCCUGA |
| | | | GAAtt (SEQ ID NO: 17) | GGta (SEQ ID NO: 18) |
| ITGA9 | 4392420 | s7553 | GGACAAUGAUGGGUUC | UGGGAACCCAUCAUUGU |
| | | | CCAtt (SEQ ID NO: 19) | CCag (SEQ ID NO: 20) |
| ITGA9 | 4457308 | s7553 | GGACAAUGAUGGGUUC | UGGGAACCCAUCAUUGU |
| | | | CCAtt (SEQ ID NO: 19) | CCag (SEQ ID NO: 20) |
| ITGA9 | AM16704 | 87036 | GGAACUUAUUCUACUU | CCAAAGUAGAAUAAGUU |
| | | | UGGtt (SEQ ID NO: 21) | CCtt (SEQ ID NO: 22) |
| ITGA9 | 4457308 | 87036 | GGAACUUAUUCUACUU | CCAAAGUAGAAUAAGUU |
| | | | UGGtt (SEQ ID NO: 21) | CCtt (SEQ ID NO: 22) |
| ITGA9 | 4390771 | s98217 | CCAUCAACCUAUACAU | AGCAUGUAUAGGUUGAU |
| | | | GCUtt (SEQ ID NO: 23) | GGtg (SEQ ID NO: 24) |
| ITGA9 | 4457308 | s98217 | CCAUCAACCUAUACAU | AGCAUGUAUAGGUUGAU |
| | | | GCUtt (SEQ ID NO: 23) | GGtg (SEQ ID NO: 24) |
| ITGA9 | 4390817 | s233797 | GAGAAAAACCGGAAAG | UCUCUUUCCGGUUUUUC |
| | | | AGAtt (SEQ ID NO: 25) | UCag (SEQ ID NO: 26) |
| ITGA9 | 4457308 | s233797 | GAGAAAAACCGGAAAG | UCUCUUUCCGGUUUUUC |
| | | | AGAtt (SEQ ID NO: 25) | UCag (SEQ ID NO: 26) |

siRNAs are commercially available from several vendors including Thermo Fisher Scientific, Qiagen, Origene, and others. The above examples are from Thermo Fisher Scientific. Additional examples include siRNAs from other companies, such as Qiagen, e.g., VLA-1 (5′-TCACAGAAGTAAAGGAGAAA-3′) (SEQ ID NO: 27), and ITGA-9 (5′-AAGAAGAAAGTC GTACTATAG-3′) (SEQ ID NO: 28). Other exemplary siRNAs include those specific to VEGFR-3 (Origene, Cat #SR301631), Ang-2 (Santa Cruz Biotechnology Inc., Cat #sc-39305; Origene, Cat #SR300199), ITGA5 (Santa Cruz Biotechnology, Inc., Cat #sc-29372; Biorbyt Ltd, Cat #orb1865790; Origene, Cat #SR320702), ITGA9 (Biorbyt Ltd, Cat #orb1865788; Origene, Cat #TR312090; Abbexa, Cat #abx920905), VEGF-A (MyBioSource, Cat #MBS8229845; Biorbyt Ltd, Cat #orb1863180), VEGF-C (Biorbyt Ltd, Cat #orb1863178; SignalChem, Cat #V812-911; Santa Cruz Biotechnology, Inc., Cat #sc-39842), and VEGF-D (Santa Cruz Biotechnology, Inc., Cat #sc-39844).

Antagonist antibodies against the targeted lymphatic formation factor can also be used to effectuate the methods described herein. Exemplary antagonist antibodies for inhibiting certain pro-lymphatic formation factors are described in Table 2.

TABLE 2
Exemplary Antibodies as Inhibitors of Pro-lymphatic Factors

| Lymphatic | | | |
|---|---|---|---|
| Formation | Clone or ID | | |
| Factor | number | Vendor | Cat # |
| VEGFR-3 | hF4-3C5 | ImClone Systems Inc. (part | |
| | | of Eli Lilly Co.) | |
| VLA-1 | Ha31/8 | BD Pharmingen, BD | 555000, |
| | | Biosciences | 555001 |
| | | Fisher Scientific (part of | BDB740046 |
| | | Thermo Fisher Scientific) | |
| VLA-1 | SAN-300 | Bausch Health Americas, | |
| | | Inc. | |
| Ang-2 | LY3127804 | Eli Lilly Co. | |
| ITGA9 | 55A2C | Chemicon | |
| VEGF-A | 2G11-2A05 | BioCell | BE0399 |
| VEGF-D | RM0007-8C35 | Santa Cruz Biotechnology, | sc-101585 |
| | | Inc. | |

Antibodies are commercially available from several vendors including Thermo Fisher Scientific, abcam, Santa Cruz Biotechnology, Inc., and others. The above examples are from ImClone Systems Inc., BD Pharmingen/Biosciences, Thermo Fisher Scientific, Chemicon, BioCell, and Santa Cruz Biotechnology, Inc. Additional exemplary antibody inhibitors of lymphangiogenesis include anti-ITGA-9 antibodies (Clone Y9A2, Clone ASP5094) (see, e.g., Emori et al. Constitutive Activation of Integrin α9 Augments Self-Directed Hyperplastic and Proinflammatory Properties of Fibroblast-like Synoviocytes of Rheumatoid Arthritis. J Immunol. 199 (10): 3427-3436 (2017 Nov. 15)).

In some embodiments, the inhibitor of lymphangiogenesis is selected from a small non-coding RNA, e.g., miR-184, miR-126, miR-31, miR-181a, miR-132, miR-194, miR-186, miR-99a, miR-92a, and miR-466; a MEK inhibitor (e.g., trametinib), an integrin inhibitor (e.g. JSM6427), an angiopoietin inhibitor (e.g. L1-10), a statin, a VEGF family inhibitor (e.g. soluble VEGFR-2, soluble VEGFR-3, a VEGF-C/VEGF-D ligand trap, Aflibercept, Bevacizumab, Brolucizumab, ranibizumab, pegaptanib sodium), and/or faricimab-svoa.

In some embodiments, the inhibitor of ocular lymphangiogenesis is formulated as eye drop, depot, bolus, inhibitor-loaded contact lens, suspension, solution, ophthalmic gel, or ointment.

### Anti-Lymphatic Formation Factors

In an alternative aspect, the inhibitor of lymphangiogenesis can be an agent that activates or stimulates an anti-lymphatic formation factor that inhibits lymphangiogenesis, as described further below. In some embodiments, the inhibitor of lymphangiogenesis is selected from the group consisting of: (1) an agent activating the expression of the anti-lymphatic formation factor; (2) an agonist antibody activating the anti-lymphatic formation factor; (3) microRNA or mimics inhibiting lymphatic formation, and (4) a small molecule activating the function of the anti-lymphatic formation factor.

In some embodiments, the inhibitor of lymphangiogenesis is selected from a small non-coding RNA, e.g., miR-184, miR-126, miR-31, miR-181a, miR-132, miR-194, miR-186, miR-99a, miR-92a, and miR-466; a MEK inhibitor (e.g., trametinib), an integrin inhibitor (e.g. JSM6427), an angiopoietin inhibitor (e.g. L1-10), a statin, a VEGF family inhibitor (e.g. soluble VEGFR-2, soluble VEGFR-3, a VEGF-C/VEGF-D ligand trap, Aflibercept, Bevacizumab, Brolucizumab, ranibizumab, pegaptanib sodium), and/or faricimab-svoa.

In some embodiments, the anti-lymphatic formation factor is selected from the group consisting of microRNA184, microRNA126, miR-31, miR-181a, microRNA132, miR-194, miR-186, miR-99a, miR-92a, and miR-466, endostatin, and a VEGF family inhibitor (e.g. soluble VEGFR-2, soluble VEGFR-3).

### Method of Treatment

In some embodiments, the method disclosed herein treats glaucoma, including a primary glaucoma, by modulating the function of Schlemm's canal (SC) in the eye. Schlemm's canal is a circular canal located in the eye's anterior chamber angle. It is responsible for draining aqueous humor, the clear fluid that circulates in the eye and maintains intraocular pressure. In some embodiments, the glaucoma is a primary glaucoma. In some embodiments, the primary glaucoma is primary open-angle glaucoma (POAG). In some embodiments, the primary glaucoma is angle-closure glaucoma. In some embodiments, the primary glaucoma is congenital glaucoma. In some embodiments, the primary glaucoma is normal tension glaucoma.

In some embodiments, the present disclosure provides a method for preventing or treating an IOP-mediated ocular disorder. In some embodiments, the IOP-mediated ocular disorder is selected from the group consisting of a secondary glaucoma, glaucomatous optic neuropathy (GON), ocular hypertension, open angle glaucoma, angle-closure glaucoma, and congenital glaucoma.

In some embodiments, the method disclosed herein inhibits adhesion of SC cells in the eye. In some embodiments, the method disclosed herein inhibits SC cell function (e.g., adhesion, proliferation, migration, tube formation) in the eye. In some embodiments, the method disclosed herein increases aqueous humor outflow from the anterior chamber of the eye.

In the method disclosed herein, the inhibitor of a pro-lymphatic factor can be administered to the eye in need thereof via any suitable route, systemically or locally. In some embodiments, the inhibitor of a pro-lymphatic factor is administered topically, via intracameral injection, via subconjunctival injection, via intravitreal injection, via suprachoroidal injection, via peribulbar injection, or via retrobulbar injection.

In some embodiments, the method disclosed herein protects the eye from a glaucomatous damage, e.g., corneal edema, retinal ganglion cell loss, and retinal neural fiber layer thinning. In some embodiments, the glaucomatous damage is corneal edema. In some embodiments, the glaucomatous damage is retinal ganglion cell loss. In some embodiments, the glaucomatous damage is retinal neural fiber layer thinning.

In some embodiments, the eye in need thereof is treated with a second therapy. In some embodiments, the second therapy is selected from the group consisting of an eye drop, an oral medication, a laser therapy, and a surgery. In some embodiments, the second therapy is selected from an eye drop that decreases the amount of fluid produced in the eye or improves the drainage of fluid from the eye, an oral medication that lowers IOP, a laser therapy (e.g., laser trabeculoplasty and laser iridotomy), and a surgery (e.g., implants, trabeculectomy). In some embodiments, the second therapy is a surgery. In some embodiments, the surgery is canaloplasty.

In one aspect, the present disclosure provides a method for treating glaucoma in an eye of a subject in need thereof, the method comprising administering to the eye a therapeutically effective amount of an inhibitor of a lymphatic formation factor associated with Schlemm's canal, wherein the lymphatic factor induces or maintains lymphangiogenesis in the eye (“a pro-lymphatic formation factor”). In some embodiments, the subject has a primary glaucoma. In some embodiments, the subject has primary open angle glaucoma. In some embodiments, the subject has primary angle-closure glaucoma. In some embodiments, the subject has primary congenital glaucoma. In some embodiments, the subject has primary normal tension glaucoma.

In another aspect, the present disclosure provides a method for preventing glaucoma in an eye of a subject in need thereof, the method comprising administering to the eye a therapeutically effective amount of an inhibitor of a pro-lymphatic formation factor. In some embodiments, the subject has a primary glaucoma. In some embodiments, the subject has primary open angle glaucoma. In some embodiments, the subject has primary angle-closure glaucoma. In some embodiments, the subject has primary congenital glaucoma. In some embodiments, the subject has primary normal tension glaucoma.

In another aspect, the present disclosure provides a method of inhibiting lymphangiogenesis in an eye of a subject suffering from elevated intraocular pressure (IOP), the method comprising administering to the eye a therapeutically effective amount of an inhibitor of a pro-lymphatic formation factor, wherein the administration of the inhibitor results in lowering IOP of the eye.

In another aspect, the present disclosure provides a method of inhibiting lymphangiogenesis in an eye of a subject at risk for the development of glaucoma, the method comprising administering to the eye a therapeutically effective amount of an inhibitor of a pro-lymphatic formation factor, wherein the administration of the inhibitor results in lowering the IOP of the eye. In some embodiments, the glaucoma is a primary glaucoma.

In another aspect, the present disclosure provides a method for increasing Schlemm's canal permeability in an eye of a subject suffering from elevated IOP, the method comprising administering to the eye a therapeutically effective amount of an inhibitor of a pro-lymphatic formation factor, wherein the administration of the inhibitor lowers the IOP of the eye.

In another aspect, the present disclosure provides a method for inhibiting Schlemm's canal cellular activity in an eye of a subject suffering from elevated IOP, the method comprising administering to the eye a therapeutically effective amount of an inhibitor of a pro-lymphatic formation factor, wherein the cellular activity is selected from the group consisting of the inhibition of cellular adhesion, inhibition of cellular proliferation, inhibition of cellular migration, or inhibition of tube formation.

In another aspect, the present disclosure provides a method for increasing aqueous humor outflow in an eye's Schlemm's canal of a subject suffering from elevated IOP, the method comprising administering to the eye a therapeutically effective amount of an inhibitor of a pro-lymphatic formation factor, wherein the administration of the results in the increased aqueous humor outflow of the Schlemm's canal and a decrease of IOP in the eye.

In another aspect, the present disclosure provides a method for reducing corneal edema in an eye of a subject in need thereof, the method comprising administering to the eye a therapeutically effective amount of an inhibitor of a pro-lymphatic formation factor, wherein the administration of the inhibitor results in the reduction of corneal edema.

In another aspect, the present disclosure provides a method for reducing retinal nerve fiber layer thinning in an eye of a subject in need thereof, the method comprising administering to the eye a therapeutically effective amount of an inhibitor of a pro-lymphatic formation factor, wherein the administration of the inhibitor results in the reduction of nerve fiber thinning.

In another aspect, the present disclosure provides a method for reducing retinal ganglion cell (RGC) death in an eye of a subject in need thereof, the method comprising administering to the eye a therapeutically effective amount of an inhibitor of a pro-lymphatic formation factor, wherein the administration of the inhibitor results in reduced retinal ganglion cell (RGC) death.

In some embodiments, the subject has a primary glaucoma selected from the group consisting of primary open angle glaucoma, primary angle-closure glaucoma, primary congenital glaucoma, and primary normal tension glaucoma. In some embodiments, the subject has primary open angle glaucoma. In some embodiments, the subject has primary angle-closure glaucoma. In some embodiments, the subject has primary congenital glaucoma. In some embodiments, the subject has primary normal tension glaucoma. In some embodiments, the subject is a human.

In some embodiments, the pro-lymphatic formation factor is a VEGF/VEGFR family member. In some embodiments, the VEGF/VEGFR family member is selected from the group consisting of VEGF-A, VEGF-B, VEGF-C, VEGF-D, VEGF-E, PIGF, VEGFR-1, VEGFR-2, and VEGFR-3. In some embodiments, the VEGF/VEGFR family member is VEGF-A. In some embodiments, the VEGF/VEGFR family member is VEGF-C. In some embodiments, the VEGF/VEGFR family member is VEGF-D. In some embodiments, the VEGF/VEGFR family member is VEGFR-3.

In some embodiments, the pro-lymphatic formation factor is an integrin family member. In some embodiments, the integrin family member is selected from the group consisting of very late antigen-1 (VLA-1), integrin alpha 5 (ITGA5), and integrin alpha 9 (ITGA9). In some embodiments, the integrin family member is VLA-1. In some embodiments, the integrin family member is ITGA5. In some embodiments, the integrin family member is ITGA9.

In some embodiments, the pro-lymphatic formation factor is an angiopoietin family member. In some embodiments, the angiopoietin family member is selected from the group consisting of angiopoietin-1 (ANGPT1), angiopoietin-2 (ANGPT2 or Ang-2), and Tie2/TEK. In some embodiments, the angiopoietin family member is Ang-2.

In some embodiments, the pro-lymphatic formation factor is selected from the group consisting of: (i) a cytokine or chemokine; (ii) an extracellular matrix protein; (iii) a transcription factor; (iv) a guidance molecule; (v) a fibroblast growth factor (FGF); (vi) a protein tyrosine phosphatase (PTP); (vii) a platelet factor member or (viii) a platelet-derived growth factor; (ix) a LEC polarity factor; (x) a Notch family member; and (xi) a combination of any of (i)-(x).

In some embodiments, the pro-lymphatic formation factor is selected from the group consisting of: (i) a cytokine/chemokine, wherein the cytokine or chemokine is selected from the group consisting of interleukin 8, interferon gamma, CCR7, and SLC; (ii) an extracellular matrix protein, wherein the extracellular matrix protein is CCBE1; (iii) a transcription factor, wherein the transcription factor is selected from the group consisting of Sox18 and Hhex; (iv) a guidance molecule, wherein the guidance molecule is selected from the group consisting of neuropilin 2 and SEMA7A; (v) an FGF, wherein the FGF is FGF-2; (vi) a protein tyrosine phosphatase (PTP), wherein the PTP is PTPN14; (vii) a member of platelet factors, wherein the platelet factor member is platelet factor 4 or (viii) a platelet-derived growth factor comprising PDGF-BB; (ix) a LEC polarity factor, wherein the LEC polarity factor is selected from the group consisting of Celsr1, Vangl2, Pdk2, and Fat4; (x) a member of Notch family; (xi) ARAF, SOS1, Apelin, KIF11, REELIN, CALCRL, GJC2 (gap junction protein gamma-2), Rasip1, or FBXL7 (F-box and leucine-rich repeat protein 7); and, (xii) a combination of any one of (i)-(xi).

In some embodiments, the inhibitor of the pro-lymphatic formation factor is selected from the group consisting of: (i) an agent capable of disrupting the pro-lymphatic formation factor's gene, thereby eliminating or reducing expression of the pro-lymphatic formation factor; (ii) an siRNA capable of degrading the mRNA of a pro-lymphatic formation factor, thus reducing expression of the pro-lymphatic factor; (iii) an antagonist antibody against the pro-lymphatic formation factor which inhibits or prevents the function of the pro-lymphatic formation factor; (iv) a small molecule capable of inhibiting or preventing the function of the pro-lymphatic formation factor; and (v) a combination of any of (i)-(iv).

In some embodiments, the inhibitor of the pro-lymphatic formation factor is administered topically, via intracameral injection, via subconjunctival injection, via intravitreal injection, via suprachoroidal injection, via peribulbar injection, or via retrobulbar injection. In some embodiments, the inhibitor of the pro-lymphatic formation factor is formulated as eye drop, depot, bolus, inhibitor-loaded contact lens, suspension, solution, ophthalmic gel, or ointment.

In some embodiments, the methods described herein further comprise treating the eye with a second therapy. In some embodiments, the second therapy is selected from the group consisting of an eye drop, an oral medication, a laser therapy, and a surgery. In some embodiments, the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

In some embodiments, the subject, prior to administration of the inhibitor, has an elevated IOP of greater than about 21 mm Hg. In some embodiments, the subject, prior to administration of the inhibitor, has an elevated IOP of greater than about 25 mm Hg. In some embodiments, the subject, prior to administration of the inhibitor, has an elevated IOP of greater than about 30 mm Hg.

In some embodiments, following administration of the inhibitor, the subject's IOP in the treated eye is reduced by at least about 2 mm Hg. In some embodiments, following administration of the inhibitor, the subject's IOP in the treated eye is reduced by at least about 5 mm Hg. In some embodiments, following administration of the inhibitor, the subject's IOP in the treated eye is reduced by at least about 10 mm Hg. In some embodiments, following administration of the inhibitor the subject's IOP in the treated eye is reduced by at least about 15 mm Hg.

In one aspect, the present disclosure provides a method for treating glaucoma in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2. In some embodiments, the subject has a primary glaucoma. In some embodiments, the subject has primary open angle glaucoma. In some embodiments, the subject has primary angle-closure glaucoma. In some embodiments, the subject has primary congenital glaucoma. In some embodiments, the subject has primary normal tension glaucoma.

In another aspect, the present disclosure provides a method for preventing glaucoma in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2. In some embodiments, the glaucoma is a primary glaucoma. In some embodiments, the glaucoma is primary open angle glaucoma. In some embodiments, the glaucoma is primary angle-closure glaucoma. In some embodiments, the glaucoma is primary congenital glaucoma. In some embodiments, the glaucoma is primary normal tension glaucoma.

In another aspect, the present disclosure provides a method of inhibiting lymphangiogenesis in an eye of a subject suffering from elevated intraocular pressure (IOP), the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in lowering IOP of the eye. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2.

In another aspect, the present disclosure provides a method of inhibiting lymphangiogenesis in an eye of a subject at risk for the development of glaucoma, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in lowering the IOP of the eye. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2. In some embodiments, the glaucoma is a primary glaucoma. In some embodiments, the glaucoma is a primary open angle glaucoma. In some embodiments, the glaucoma is primary angle-closure glaucoma. In some embodiments, the glaucoma is primary congenital glaucoma. In some embodiments, the glaucoma is primary normal tension glaucoma.

In another aspect, the present disclosure provides a method for increasing Schlemm's canal permeability in an eye of a subject suffering from elevated IOP, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor lowers the TOP of the eye. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2.

In another aspect, the present disclosure provides a method for inhibiting Schlemm's canal cellular activity in an eye of a subject suffering from elevated IOP, the method comprising administering to the eye a therapeutically effective amount of an inhibitor of a pro-lymphatic formation factor selected from the group consisting of VEGFR-3, VLA-1, ITGA5, and Ang-2, wherein the cellular activity is selected from the group consisting of the inhibition of cellular adhesion, inhibition of cellular proliferation, inhibition of cellular migration, or inhibition of tube formation. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2.

In another aspect, the present disclosure provides a method for increasing aqueous humor outflow in an eye's Schlemm's canal of a subject suffering from elevated IOP, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in the increased aqueous humor outflow of the Schlemm's canal and a decrease of IOP in the eye. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2.

In another aspect, the present disclosure provides a method for reducing corneal edema in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in the reduction of corneal edema. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2.

In another aspect, the present disclosure provides a method for reducing retinal nerve fiber layer (RNFL) thinning in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in the reduction of RNFL thinning. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2.

In another aspect, the present disclosure provides a method for reducing retinal ganglion cell (RGC) death in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in reduced RGC death. In some embodiments, the pro-lymphatic formation factor is VEGFR-3. In some embodiments, the pro-lymphatic formation factor is VLA-1. In some embodiments, the pro-lymphatic formation factor is ITGA5. In some embodiments, the pro-lymphatic formation factor is Ang-2.

In some embodiments, the subject has a primary glaucoma selected from the group consisting of primary open angle glaucoma, primary angle-closure glaucoma, primary congenital glaucoma, and primary normal tension glaucoma, the subject has primary open angle glaucoma. In some embodiments, the subject has primary angle-closure glaucoma. In some embodiments, the subject has primary congenital glaucoma. In some embodiments, the subject has primary normal tension glaucoma. In some embodiments, the subject is a human.

In some embodiments, the inhibitor of the pro-lymphatic formation factor is selected from the group consisting of: (i) an agent capable of disrupting the pro-lymphatic formation factor's gene, thereby eliminating or reducing expression of the pro-lymphatic formation factor; (ii) an siRNA capable of degrading the mRNA of a pro-lymphatic formation factor, thus reducing expression of the pro-lymphatic factor; (iii) an antagonist antibody against the pro-lymphatic formation factor which inhibits or prevents the function of the pro-lymphatic formation factor; (iv) a small molecule capable of inhibiting or preventing the function of the pro-lymphatic formation factor; and (v) a combination of any of (i)-(iv).

In some embodiments, the inhibitor of the pro-lymphatic formation factor is administered topically, via intracameral injection, via subconjunctival injection, via intravitreal injection, via suprachoroidal injection, via peribulbar injection, or via retrobulbar injection. In some embodiments, the inhibitor of the pro-lymphatic formation factor is formulated as eye drop, depot, bolus, inhibitor-loaded contact lens, suspension, solution, ophthalmic gel, or ointment.

In some embodiments, the methods described herein further comprise treating the eye with a second therapy. In some embodiments, the second therapy is selected from the group consisting of an eye drop, an oral medication, a laser therapy, and a surgery. In some embodiments, the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

In some embodiments, the subject, prior to administration of the inhibitor, has an elevated IOP of greater than about 21 mm Hg. In some embodiments, the subject, prior to administration of the inhibitor, has an elevated IOP of greater than about 25 mm Hg. In some embodiments, the subject, prior to administration of the inhibitor, has an elevated IOP of greater than about 30 mm Hg.

In some embodiments, following administration of the inhibitor, the subject's IOP in the treated eye is reduced by at least about 1 mm Hg. In some embodiments, following administration of the inhibitor, the subject's IOP in the treated eye is reduced by at least about 2 mm Hg. In some embodiments, following administration of the inhibitor, the subject's IOP in the treated eye is reduced by at least about 5 mm Hg. In some embodiments, following administration of the inhibitor, the subject's IOP in the treated eye is reduced by at least about 10 mm Hg. In some embodiments, following administration of the inhibitor the subject's IOP in the treated eye is reduced by at least about 15 mm Hg.

In some embodiments, the pro-lymphatic formation factor is VEGFR-3. In some embodiments, the pro-lymphatic formation factor is VLA-1. In some embodiments, the pro-lymphatic formation factor is ITAG5. In some embodiments, the pro-lymphatic formation factor is Ang-2.

In some embodiments, the inhibitor is an siRNA. In some embodiments, the inhibitor is an antagonist antibody. In some embodiments, the inhibitor disrupts the pro-lymphatic formation factor's gene's expression. In some embodiments, the inhibitor that disrupts the pro-lymphatic formation factor's gene's expression is a gene editing composition. In some embodiments, the gene editing composition comprises CRISPR/Cas9 and gRNA. In some embodiments, the subject is human.

In an alternative aspect, the present disclosure provides a method for treating primary glaucoma in an eye of a subject in need thereof, the method comprising administering to the eye a therapeutically effective amount an agent that activates or stimulates a lymphatic factor that inhibits or prevents lymphangiogenesis in the eye (“an anti-lymphatic formation factor”).

In some embodiments, the subject has a primary glaucoma selected from the group consisting of primary open angle glaucoma, primary angle-closure glaucoma, primary congenital glaucoma, and primary normal tension glaucoma, the subject has primary open angle glaucoma. In some embodiments, the subject has primary angle-closure glaucoma. In some embodiments, the subject has primary congenital glaucoma. In some embodiments, the subject has primary normal tension glaucoma. In some embodiments, the subject is a human.

In another aspect, the present disclosure provides a method for preventing primary glaucoma in an eye of a subject in need thereof, the method comprising administering to the eye a therapeutically effective amount of an anti-lymphatic formation factor.

In another aspect, the present disclosure provides a method of inhibiting lymphangiogenesis in an eye of a subject suffering from elevated intraocular pressure (IOP), the method comprising administering to the eye a therapeutically effective amount of an agent that activates or stimulates an anti-lymphatic formation factor, wherein the administration of the agent results in lowering IOP of the eye.

In another aspect, the present disclosure provides a method of inhibiting lymphangiogenesis in an eye of a subject at risk for the development of primary glaucoma, the method comprising administering to the eye a therapeutically effective amount of an agent that activates or stimulates anti-lymphatic formation factor, wherein the administration of the agent results in lowering the TOP of the eye.

In another aspect, the present disclosure provides a method for increasing Schlemm's canal permeability in an eye of a subject suffering from elevated IOP, the method comprising administering to the eye a therapeutically effective amount of an agent that activates or stimulates an anti-lymphatic formation factor, wherein the administration of the agent lowers the IOP of the eye.

In another aspect, the present disclosure provides a method for inhibiting Schlemm's canal cellular activity in an eye of a subject suffering from elevated IOP, the method comprising administering to the eye a therapeutically effective amount of an agent that activates or stimulates an anti-lymphatic formation factor, wherein the cellular activity is selected from the group consisting of the inhibition of cellular adhesion, inhibition of cellular proliferation, inhibition of cellular migration, or inhibition of tube formation.

In another aspect, the present disclosure provides a method for increasing aqueous humor outflow in an eye's Schlemm's canal of a subject suffering from elevated IOP, the method comprising administering to the eye a therapeutically effective amount of an agent that activates or stimulates an anti-lymphatic formation factor, wherein the administration of the agent results in the increased aqueous humor outflow of the Schlemm's canal and a decrease of IOP in the eye.

In another aspect, the present disclosure provides a method for reducing corneal edema in an eye of a subject in need thereof, the method comprising administering to the eye a therapeutically effective amount of an inhibitor of an agent that activates or stimulates an anti-lymphatic formation factor, wherein the administration of the agent results in the reduction of corneal edema.

In another aspect, the present disclosure provides a method for reducing retinal nerve fiber layer (RNFL) thinning in an eye of a subject in need thereof, the method comprising administering to the eye a therapeutically effective amount of an agent that activates or stimulates an anti-lymphatic formation factor, wherein the administration of the agent in the reduction of RNFL thinning.

In another aspect, the present disclosure provides a method for reducing retinal ganglion cell (RGC) death in an eye of a subject in need thereof, the method comprising administering to the eye a therapeutically effective amount of an agent that activates or stimulates an anti-lymphatic formation factor, wherein the administration of the agent results in reduced RGC death.

In some embodiments of the aspects described herein, the subject has a primary glaucoma selected from the group consisting of primary open angle glaucoma, primary angle-closure glaucoma, primary congenital glaucoma, and primary normal tension glaucoma. the subject has primary open angle glaucoma. In some embodiments, the subject has primary angle-closure glaucoma. In some embodiments, the subject has primary congenital glaucoma. In some embodiments, the subject has primary normal tension glaucoma. In some embodiments, the subject is a human.

In some embodiments, the agent is administered topically, via intracameral injection, via subconjunctival injection, via intravitreal injection, via suprachoroidal injection, via peribulbar injection, or via retrobulbar injection. In some embodiments, the agent is formulated as eye drop, depot, bolus, inhibitor-loaded contact lens, suspension, solution, ophthalmic gel, or ointment.

In some embodiments, the methods described herein further comprise treating the eye with a second therapy. In some embodiments, the second therapy is selected from the group consisting of an eye drop, an oral medication, a laser therapy, and a surgery. In some embodiments, the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

In some embodiments, the subject, prior to administration of the agent, has an elevated IOP of greater than about 21 mm Hg. In some embodiments, the subject, prior to administration of the agent, has an elevated IOP of greater than about 25 mm Hg. In some embodiments, the subject, prior to administration of the agent, has an elevated IOP of greater than about 30 mm Hg.

In some embodiments, following administration of the agent, the subject's IOP in the treated eye is reduced by at least about 2 mm Hg. In some embodiments, following administration of the agent, the subject's IOP in the treated eye is reduced by at least about 5 mm Hg. In some embodiments, following administration of the agent, the subject's IOP in the treated eye is reduced by at least about 10 mm Hg. In some embodiments, following administration of the agent the subject's IOP in the treated eye is reduced by at least about 15 mm Hg.

In some embodiments, the agent administered is selected from the group consisting of: (i) an anti-lymphatic formation factor; (ii) an agent that activates the expression of the anti-lymphatic formation factor; (iii) an agonist antibody activating the anti-lymphatic formation factor; (iv) a microRNA or mimics inhibiting lymphatic formation, and (v) a small molecule activating the function of the anti-lymphatic formation factor.

In some embodiments, the anti-lymphatic formation factor is selected from the group consisting of: microRNA184, microRNA126, miR-31, miR-181a, microRNA132, miR-194, miR-186, miR-99a, miR-92a, and miR-466, endostatin, and a VEGF family inhibitor (e.g. soluble VEGFR-2, soluble VEGFR-3).

### Pharmaceutical Compositions

In one aspect, the invention provides pharmaceutical ophthalmological compositions comprising an inhibitor of a pro-lymphatic factor, as described herein, and a pharmaceutically acceptable carrier. The pharmaceutical composition comprising a pro-lymphatic factor inhibitor is useful for treating glaucoma, including a primary glaucoma or an IOP-mediated disorder. Such pharmaceutical compositions are formulated based on the mode of delivery. One example is compositions formulated for direct delivery to the eye. In some embodiments, the delivery is topical and the mode of delivery is selected from the group consisting of eye drops, intracameral injection, subconjunctival injection, intravitreal injection, suprachoroidal injection, peribulbar injection, and retrobulbar injection. In some embodiments, the pharmaceutical ophthalmological composition is a liquid. In alternative embodiments, the pharmaceutical ophthalmological compositions comprising an activator of an anti-lymphatic factor, as described herein, and a pharmaceutically acceptable carrier.

The dosage administered will, of course, vary depending upon known factors such as the pharmacodynamic characteristics of the particular agent, and its mode and route of administration; age, health, nature and extent of symptoms, kind of concurrent treatment, frequency of treatment, and the effect desired. The pharmaceutical compositions featured herein are administered in dosages sufficient to inhibit expression of a pro-lymphatic factor.

In some embodiments, the pharmaceutical ophthalmological composition comprises at least one siRNA. In some embodiments, a suitable dose of siRNA will be in the range of 0.000001 to 100.0 milligrams per eye of the recipient per a single dose, including 0.000001, 0.00001, 0.0001, 0.0005, 0.001, 0.005, 0.01, 0.05, 0.1, 0.5, 1, 5, 10, 50, or 100 milligrams. In some embodiments, the pharmaceutical composition is comprised within a volume of between about 1 μL to about 500 μL, including 1.0, 3.0, 5.0, 10.0, 15.0, 20.0, 25.0, 50.0, 100.0, 200.0, 250.0, 300.0, or 500.0 μL. For example, an siRNA can be administered at 0.05 μg/eye/50 μL, 0.25 μg/eye/50 μL, 0.5 μg/eye/50 μL, 5 μg/eye/50 μL, 50 μg/eye/50 μL, 100 μg/eye/50 μL, 250 μg/eye/50 μL, 500 μg/eye/50 μL, 750 μg/eye/50 μL, 950 μg/eye/50 μL, 1000 μg/eye/50 μL, per a single dose. In some embodiments, the pharmaceutical composition volume is limited by the mode of delivery (e.g., 100 μL for intravitreal injection).

In some embodiments, the pharmaceutical ophthalmological composition comprises at least one antibody. In some embodiments, the pharmaceutical composition is in a dosage form that contains from about 0.01 mg to about 2000 mg, from about 0.1 mg to about 1000 mg, from about from about 1 mg to about 800 mg, from about 10 mg to about 600 mg, or from about 100 mg to about 500 mg of the active compound and optionally from about 0.01 mg to about 1000 mg, from about 0.1 mg to about 500 mg, from about 1 mg to about 200 mg, or from about 10 mg to about 100 mg of an additional active agent in a unit dosage form. Examples are dosage forms with at least 0.01, 0.1, 1, 5, 10, 25, 50, 100, 200, 250, 300, 400, 500, 600, 700, or 750 mg of active compound, or its salt. For example, an antibody can be administered at 50 μg/eye/50 μL, 100 μg/eye/50 μL, 250 μg/eye/50 μL, 500 μg/eye/50 μL, 1000 μg/eye/50 μL, per a single dose. In some embodiments, the pharmaceutical composition volume is limited by the mode of delivery (e.g., 100 μL for intravitreal injection).

The pharmaceutical compositions described herein may be administered once daily, once weekly, twice weekly, once monthly, once in 2 months, once in 3 months, or once in 6 months, or the pharmaceutical composition may be administered as two, three, or more sub-doses at appropriate intervals throughout a time period or even using continuous infusion or delivery through a controlled release formulation. In that case, the dosage of a pro-lymphatic factor inhibitor contained in each sub-dose must be correspondingly smaller in order to achieve the total dosage in a certain time period. The dosage unit can also be compounded for delivery over several days, e.g., using a conventional sustained release formulation which provides sustained release of a pro-lymphatic factor inhibitor over a several day or month period. Sustained release formulations are well known in the art and are particularly useful for delivery of agents at a particular site, such as could be used with the agents of the present invention. In this embodiment, the dosage unit contains a corresponding multiple of the single dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual siRNAs or antibodies or other compound encompassed by the invention can be made using conventional methodologies or on the basis of in vivo testing using an appropriate animal model, as described elsewhere herein.

Carriers include sterile excipients and diluents and must be of sufficiently high purity and sufficiently low toxicity to ocular tissues to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, glidents, lubricants, preservatives, stabilizers, surfactants, and wetting agents. Some carriers may be listed in more than one class. Optional active agents may be included in a pharmaceutical composition, which do not substantially interfere with the activity of the ocular lymphangiogenesis inhibitor of the present invention.

### Embodiments

1. A method for treating glaucoma in an eye of a subject in need thereof, the method comprising administering to the eye a therapeutically effective amount of an inhibitor of a lymphatic formation factor associated with the Schlemm's canal, wherein the lymphatic factor induces or maintains lymphangiogenesis in the eye (“a pro-lymphatic formation factor”).

2. The method of embodiment 1, wherein the subject has a primary glaucoma.

3. The method of embodiment 2, wherein the subject has primary open angle glaucoma.

4. The method of embodiment 2, wherein the subject has primary angle-closure glaucoma.

5. The method of embodiment 2, wherein the subject has primary congenital glaucoma.

6. The method of embodiment 2, wherein the subject has primary normal tension glaucoma.

7. A method for preventing glaucoma in an eye of a subject in need thereof, the method comprising administering to the eye a therapeutically effective amount of an inhibitor of a pro-lymphatic formation factor.

8. A method of inhibiting lymphangiogenesis in an eye of a subject at risk for the development of glaucoma, the method comprising administering to the eye a therapeutically effective amount of an inhibitor of a pro-lymphatic formation factor, wherein the administration of the inhibitor results in lowering IOP of the eye.

9. A method of inhibiting lymphangiogenesis in an eye of a subject suffering from elevated intraocular pressure (IOP), the method comprising administering to the eye a therapeutically effective amount of an inhibitor of a pro-lymphatic formation factor, wherein the administration of the inhibitor results in lowering IOP of the eye.

10. A method for increasing Schlemm's canal permeability in an eye of a subject suffering from elevated IOP, the method comprising administering to the eye a therapeutically effective amount of an inhibitor of a pro-lymphatic formation factor, wherein the administration of the inhibitor lowers the IOP of the eye.

11. A method for inhibiting Schlemm's canal cellular activity in an eye of a subject suffering from elevated IOP, the method comprising administering to the eye a therapeutically effective amount of an inhibitor of a pro-lymphatic formation factor, wherein the cellular activity is selected from the group consisting of the inhibition of cellular adhesion, inhibition of cellular proliferation, inhibition of cellular migration, or inhibition of tube formation.

12. A method for increasing aqueous humor outflow in an eye's Schlemm's canal of a subject suffering from elevated IOP, the method comprising administering to the eye a therapeutically effective amount of an inhibitor of a pro-lymphatic formation factor, wherein the administration of the results in the increased aqueous humor outflow of the Schlemm's canal and a decrease of IOP in the eye.

13 A method for reducing corneal edema in an eye of a subject in need thereof, the method comprising administering to the eye a therapeutically effective amount of an inhibitor of a pro-lymphatic formation factor, wherein the administration of the inhibitor results in the reduction of corneal edema.

14. A method for reducing retinal nerve fiber layer thinning in an eye of a subject in need thereof, the method comprising administering to the eye a therapeutically effective amount of an inhibitor of a pro-lymphatic formation factor, wherein the administration of the inhibitor results in the reduction of nerve fiber thinning.

15. A method for reducing retinal ganglion cell (RGC) death in an eye of a subject in need thereof, the method comprising administering to the eye a therapeutically effective amount of an inhibitor of a pro-lymphatic formation factor, wherein the administration of the inhibitor results in reduced retinal ganglion cell (RGC) death.

16. The method of embodiments 9-15, wherein the subject has a primary glaucoma.

17. The method of embodiment 16, wherein the subject has primary open angle glaucoma.

18. The method of embodiment 16, wherein the subject has primary angle-closure glaucoma.

19. The method of embodiment 16, wherein the subject has primary congenital glaucoma.

20. The method of embodiment 16, wherein the subject has primary normal tension glaucoma.

21. The method of embodiments 1-20, wherein the subject is a human.

22. The method of embodiments 1-21, wherein the pro-lymphatic formation factor is a VEGF/VEGFR family member.

23. The method of embodiment 22, wherein the VEGF/VEGFR family member is selected from the group consisting of VEGF-A, VEGF-B, VEGF-C, VEGF-D, VEGF-E, PIGF, VEGFR-1, VEGFR-2, and VEGFR-3.

24. The method of embodiment 23, wherein the VEGF/VEGFR family member is VEGF-A.

25. The method of embodiment 23, wherein the VEGF/VEGFR family member is VEGF-C.

26. The method of embodiment 23, wherein the VEGF/VEGFR family member is VEGF-D.

27. The method of embodiment 23, wherein the VEGF/VEGFR family member is VEGFR-3.

28. The method of embodiments 1-21, wherein the pro-lymphatic formation factor is an integrin family member.

29. The method of embodiment 28, wherein the integrin family member is selected from the group consisting of very late antigen-1 (VLA-1), integrin alpha 5 (ITGA5), and integrin alpha 9 (ITGA9).

30. The method of embodiment 29, wherein the integrin family member is VLA-1.

31. The method of embodiment 29, wherein the integrin family member is ITGA5.

32. The method of embodiment 29, wherein the integrin family member is ITGA9.

33. The method of embodiments 1-21, wherein the pro-lymphatic formation factor is an angiopoietin family member.

34. The method of embodiment 33, wherein the angiopoietin family member is selected from the group consisting of angiopoietin-1 (ANGPT1), angiopoietin-2 (ANGPT2 or Ang-2), and Tie2/TEK.

35. The method of embodiment 34, wherein the angiopoietin family member is Ang-2.

36. The method of embodiments 1-21, wherein the pro-lymphatic formation factor is selected from the group consisting of:
(i) a cytokine or chemokine;
(ii) an extracellular matrix protein;
(iii) a transcription factor;
(iv) a guidance molecule;
(v) a fibroblast growth factor (FGF);
(vi) a protein tyrosine phosphatase (PTP);
(vii) a platelet factor member or (viii) a platelet-derived growth factor;
(ix) a LEC polarity factor;
(x) a Notch family member; and
(xi) a combination of any of (i)-(xi).

37. The method of embodiments 1-21, wherein the pro-lymphatic formation factor is selected from the group consisting of:
(i) a cytokine/chemokine, wherein the cytokine or chemokine is selected from the group consisting of interleukin 8, interferon gamma, CCR7, and SLC;
(ii) an extracellular matrix protein, wherein the extracellular matrix protein is CCBE1; (iii) a transcription factor, wherein the transcription factor is selected from the group consisting of Sox18 and Hhex;
(iv) a guidance molecule, wherein the guidance molecule is selected from the group consisting of neuropilin 2 and SEMA7A;
(v) an FGF, wherein the FGF is FGF-2;
(vi) a protein tyrosine phosphatase (PTP), wherein the PTP is PTPN14;
(vii) a member of platelet factors, wherein the platelet factor member is platelet factor 4 or (viii) a platelet-derived growth factor comprising PDGF-BB;
(ix) a LEC polarity factor, wherein the LEC polarity factor is selected from the group consisting of Celsr1, Vangl2, Pdk2, and Fat4;
(x) a member of Notch family;
(xi) ARAF, SOS1, Apelin, KIF11, REELIN, CALCRL, GJC2 (gap junction protein gamma-2), Rasip1, or FBXL7 (F-box and leucine-rich repeat protein 7); and,
(xii) a combination of any one of (i)-(xi).

38. The method of embodiments 1-37, wherein the inhibitor of the pro-lymphatic formation factor is selected from the group consisting of:
(i) an agent capable of disrupting the pro-lymphatic formation factor's gene, thereby eliminating or reducing expression of the pro-lymphatic formation factor;
(ii) an siRNA capable of degrading the mRNA of a pro-lymphatic formation factor, thus reducing expression of the pro-lymphatic factor;
(iii) an antagonist antibody against the pro-lymphatic formation factor which inhibits or prevents the function of the pro-lymphatic formation factor;
(iv) a small molecule capable of inhibiting or preventing the function of the pro-lymphatic formation factor; and
(v) a combination of any of (i)-(iv).

39. The method of embodiments 1-38, wherein the inhibitor of the pro-lymphatic formation factor is administered topically, via intracameral injection, via subconjunctival injection, via intravitreal injection, via suprachoroidal injection, via peribulbar injection, or via retrobulbar injection.

40. The method of embodiments 1-37, wherein the inhibitor of the pro-lymphatic formation factor is formulated as eye drop, depot, bolus, inhibitor-loaded contact lens, suspension, solution, ophthalmic gel, or ointment.

41. The method of embodiments 1-40, further comprising treating the eye with a second therapy.

42. The method of embodiment 41, wherein the second therapy is selected from the group consisting of an eye drop, an oral medication, a laser therapy, and a surgery.

43. The method of embodiments 41 or 42, wherein the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

44. The method of embodiments 1-43, wherein the subject, prior to administration of the inhibitor, has an elevated IOP of greater than about 21 mm Hg.

45. The method of embodiments 1-43, wherein the subject, prior to administration of the inhibitor, has an elevated IOP of greater than about 25 mm Hg.

46. The method of embodiments 1-43, wherein the subject, prior to administration of the inhibitor, has an elevated IOP of greater than about 30 mm Hg.

47. The method of embodiment 1-43, wherein, following administration of the inhibitor, the subject's IOP in the treated eye is reduced by at least about 2 mm Hg.

48. The method of embodiment 1-43, wherein, following administration of the inhibitor, the subject's IOP in the treated eye is reduced by at least about 5 mm Hg.

49. The method of embodiment 1-43, wherein, following administration of the inhibitor, the subject's IOP in the treated eye is reduced by at least about 10 mm Hg.

50. The method of embodiment 1-43, wherein, following administration of the inhibitor the subject's IOP in the treated eye is reduced by at least about 15 mm Hg.

51. A method for treating glaucoma in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2.

52. The method of embodiment 51, wherein the subject has a primary glaucoma.

53. The method of embodiment 52, wherein the subject has primary open angle glaucoma.

54. The method of embodiment 52, wherein the subject has primary angle-closure glaucoma.

55. The method of embodiment 52, wherein the subject has primary congenital glaucoma.

56. The method of embodiment 52, wherein the subject has primary normal tension glaucoma.

57. A method for preventing glaucoma in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2.

58. A method of inhibiting lymphangiogenesis in an eye of a subject at risk for the development of glaucoma, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in lowering IOP of the eye.

59. A method of inhibiting lymphangiogenesis in an eye of a subject suffering from elevated intraocular pressure (IOP), the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in lowering IOP of the eye.

60. A method for increasing Schlemm's canal permeability in an eye of a subject suffering from elevated IOP, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor lowers the TOP of the eye.

61. A method for inhibiting Schlemm's canal cellular activity in an eye of a subject suffering from elevated IOP, the method comprising administering to the eye a therapeutically effective amount of an inhibitor of a pro-lymphatic formation factor selected from the group consisting of VEGFR-3, VLA-1, ITGA5, and Ang-2, wherein the cellular activity is selected from the group consisting of the inhibition of cellular adhesion, inhibition of cellular proliferation, inhibition of cellular migration, or inhibition of tube formation.

62. A method for increasing aqueous humor outflow in an eye's Schlemm's canal of a subject suffering from elevated IOP, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in the increased aqueous humor outflow of the Schlemm's canal and a decrease of IOP in the eye.

63. A method for reducing corneal edema in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in the reduction of corneal edema.

64. A method for reducing retinal nerve fiber layer (RNFL) thinning in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in the reduction of RNFL thinning.

65. A method for reducing retinal ganglion cell (RGC) death in an eye of a subject in need thereof, the method comprising administering to the eye a therapeutically effective amount of an inhibitor of a pro-lymphatic formation factor selected from the group consisting of VEGFR-3, VLA-1, ITGA5, and Ang-2, wherein the administration of the inhibitor results in reduced RGC death.

66. The method of embodiments 59-66, wherein the subject has a primary glaucoma.

67. The method of embodiment 67, wherein the subject has primary open angle glaucoma.

68. The method of embodiment 67, wherein the subject has primary angle-closure glaucoma.

69. The method of embodiment 67, wherein the subject has primary congenital glaucoma.

70. The method of embodiment 67, wherein the subject has primary normal tension glaucoma.

71. The method of embodiments 57-70, wherein the subject is a human.

72. The method of embodiments 51-71, wherein the inhibitor of the pro-lymphatic formation factor is selected from the group consisting of:
(i) an agent capable of disrupting the pro-lymphatic formation factor's gene, thereby eliminating or reducing expression of the pro-lymphatic formation factor;
(ii) an siRNA capable of degrading the mRNA of a pro-lymphatic formation factor, thus reducing expression of the pro-lymphatic factor;
(iii) an antagonist antibody against the pro-lymphatic formation factor which inhibits or prevents the function of the pro-lymphatic formation factor;
(iv) a small molecule capable of inhibiting or preventing the function of the pro-lymphatic formation factor; and
(v) a combination of any of (i)-(iv).

73. The method of embodiments 51-72, wherein the inhibitor of the pro-lymphatic formation factor is administered topically, via intracameral injection, via subconjunctival injection, via intravitreal injection, via suprachoroidal injection, via peribulbar injection, or via retrobulbar injection.

74. The method of embodiments 51-73, wherein the inhibitor of the pro-lymphatic formation factor is formulated as eye drop, depot, bolus, inhibitor-loaded contact lens, suspension, solution, ophthalmic gel, or ointment.

75. The method of embodiments 51-74, further comprising treating the eye with a second therapy.

76. The method of embodiment 75, wherein the second therapy is selected from the group consisting of an eye drop, an oral medication, a laser therapy, and a surgery.

77. The method of embodiments 75 or 76, wherein the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

78. The method of embodiments 51-77, wherein the subject, prior to administration of the inhibitor, has an elevated IOP of greater than about 21 mm Hg.

79. The method of embodiments 51-77, wherein the subject, prior to administration of the inhibitor, has an elevated IOP of greater than about 25 mm Hg.

80. The method of embodiments 51-77, wherein the subject, prior to administration of the inhibitor, has an elevated IOP of greater than about 30 mm Hg.

81. The method of embodiment 51-80, wherein following administration of the inhibitor, the subject's IOP in the treated eye is reduced by at least about 2 mm Hg.

82. The method of embodiment 51-80, wherein following administration of the inhibitor, the subject's IOP in the treated eye is reduced by at least about 5 mm Hg.

83. The method of embodiment 51-80, wherein following administration of the inhibitor, the subject's IOP in the treated eye is reduced by at least about 10 mm Hg.

84. The method of embodiment 51-80, wherein following administration of the inhibitor the subject's IOP in the treated eye is reduced by at least about 15 mm Hg.

85. The method of embodiments 51-84, wherein the pro-lymphatic formation factor is VEGFR-3.

86. The method of embodiments 51-84, wherein the pro-lymphatic formation factor is VLA-1.

87. The method of embodiments 51-80, wherein the pro-lymphatic formation factor is ITAG5.

88. The method of embodiments 51-84, wherein the pro-lymphatic formation factor is Ang-2.

89. The method of embodiments 1-88, wherein the inhibitor is an siRNA.

90. The method of embodiments 1-88, wherein the inhibitor is an antagonist antibody.

91. The method of embodiments 1-88, wherein the inhibitor disrupts the pro-lymphatic formation factor's gene's expression.

92. The method of embodiments 1-91, wherein the subject is human.

93. A method for treating glaucoma in an eye of a subject in need thereof, the method comprising administering to the eye a therapeutically effective amount an agent that activates or stimulates a lymphatic factor that inhibits or prevents lymphangiogenesis in the eye (“an anti-lymphatic formation factor”).

94. The method of embodiment 93, wherein the subject has primary glaucoma.

95. The method of embodiment 94, wherein the subject has primary open angle glaucoma.

96. The method of embodiment 94, wherein the subject has primary angle-closure glaucoma.

97. The method of embodiment 94, wherein the subject has primary congenital glaucoma.

98. The method of embodiment 94, wherein the subject has primary normal tension glaucoma.

99. A method for preventing glaucoma in an eye of a subject in need thereof, the method comprising administering to the eye a therapeutically effective amount of an anti-lymphatic formation factor.

100. A method of inhibiting lymphangiogenesis in an eye of a subject at risk for the development of primary glaucoma, the method comprising administering to the eye a therapeutically effective amount of an agent that activates or stimulates anti-lymphatic formation factor, wherein the administration of the agent results in lowering IOP of the eye.

101. A method of inhibiting lymphangiogenesis in an eye of a subject suffering from elevated intraocular pressure (IOP), the method comprising administering to the eye a therapeutically effective amount of an agent that activates or stimulates an anti-lymphatic formation factor, wherein the administration of the agent results in lowering IOP of the eye.

102. A method for increasing Schlemm's canal permeability in an eye of a subject suffering from elevated IOP, the method comprising administering to the eye a therapeutically effective amount of an agent that activates or stimulates an anti-lymphatic formation factor, wherein the administration of the agent lowers the TOP of the eye.

103. A method for inhibiting Schlemm's canal cellular activity in an eye of a subject suffering from elevated IOP, the method comprising administering to the eye a therapeutically effective amount of an agent that activates or stimulates an anti-lymphatic formation factor, wherein the cellular activity is selected from the group consisting of the inhibition of cellular adhesion, inhibition of cellular proliferation, inhibition of cellular migration, or inhibition of tube formation.

104. A method for increasing aqueous humor outflow in an eye's Schlemm's canal of a subject suffering from elevated IOP, the method comprising administering to the eye a therapeutically effective amount of an agent that activates or stimulates an anti-lymphatic formation factor, wherein the administration of the agent results in the increased aqueous humor outflow of the Schlemm's canal and a decrease of IOP in the eye.

105. A method for reducing corneal edema in an eye of a subject in need thereof, the method comprising administering to the eye a therapeutically effective amount of an inhibitor of an agent that activates or stimulates an anti-lymphatic formation factor, wherein the administration of the agent results in the reduction of corneal edema.

106. A method for reducing retinal nerve fiber layer (RNFL) thinning in an eye of a subject in need thereof, the method comprising administering to the eye a therapeutically effective amount of an agent that activates or stimulates an anti-lymphatic formation factor, wherein the administration of the agent in the reduction of RNFL thinning.

107. A method for reducing retinal ganglion cell (RGC) death in an eye of a subject in need thereof, the method comprising administering to the eye a therapeutically effective amount of an agent that activates or stimulates an anti-lymphatic formation factor, wherein the administration of the agent results in reduced RGC death.

108. The method of embodiments 100-107, wherein the subject has primary glaucoma.

109. The method of embodiment 108, wherein the subject has primary open angle glaucoma.

110. The method of embodiment 108, wherein the subject has primary angle-closure glaucoma.

111. The method of embodiment 108, wherein the subject has primary congenital glaucoma.

112. The method of embodiment 108, wherein the subject has primary normal tension glaucoma.

113. The method of embodiments 93-112, wherein the subject is a human.

114. The method of embodiments 93-113, wherein the agent is administered topically, via intracameral injection, via subconjunctival injection, via intravitreal injection, via suprachoroidal injection, via peribulbar injection, or via retrobulbar injection.

115. The method of embodiments 93-114, wherein the agent is formulated as eye drop, depot, bolus, inhibitor-loaded contact lens, suspension, solution, ophthalmic gel, or ointment.

116. The method of embodiments 93-115, further comprising treating the eye with a second therapy.

117. The method of embodiment 116, wherein the second therapy is selected from the group consisting of an eye drop, an oral medication, a laser therapy, and a surgery.

118. The method of embodiments 116 or 117, wherein the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

119. The method of embodiments 93-118, wherein the subject, prior to administration of the agent, has an elevated IOP of greater than about 21 mm Hg.

120. The method of embodiments 93-118, wherein the subject, prior to administration of the agent, has an elevated IOP of greater than about 25 mm Hg.

121. The method of embodiments 93-118, wherein the subject, prior to administration of the agent, has an elevated IOP of greater than about 30 mm Hg.

122. The method of embodiment 93-121, wherein, following administration of the agent, the subject's IOP in the treated eye is reduced by at least about 2 mm Hg.

123. The method of embodiment 93-121, wherein, following administration of the agent, the subject's IOP in the treated eye is reduced by at least about 5 mm Hg.

124. The method of embodiment 93-121, wherein, following administration of the agent, the subject's IOP in the treated eye is reduced by at least about 10 mm Hg.

125. The method of embodiment 93-121, wherein, following administration of the agent the subject's IOP in the treated eye is reduced by at least about 15 mm Hg.

126. The method of embodiments 93-125, wherein the agent administered is selected from the group consisting of:
(i) an anti-lymphatic formation factor;
(ii) an agent that activates the expression of the anti-lymphatic formation factor;
(iii) an agonist antibody activating the anti-lymphatic formation factor;
(iv) a microRNA or mimics inhibiting lymphatic formation, and
(v) a small molecule activating the function of the anti-lymphatic formation factor.

127. The method of embodiments 93-126, wherein the anti-lymphatic formation factor is selected from the group consisting of: microRNA184, microRNA126, miR-31, miR-181a, microRNA132, miR-194, miR-186, miR-99a, miR-92a, and miR-466, endostatin, and a VEGF family inhibitor (e.g. soluble VEGFR-2, soluble VEGFR-3).

### EXAMPLES

### Example 1. VEGFR-3 Blockade Lowers IOP and Protects the Eye from Glaucomatous Damage

This example illustrates that the anti-VEGFR-3 treatment via siRNAs significantly lowered IOP and protected the eye from glaucomatous damage, as demonstrated in a mouse model of glaucoma.

### Methods

### Materials

Intraocular hypertension was induced in normal eyes by laser photocoagulation of the episcleral veins. Anti-VEGFR-3 siRNA (Sequence (5′->3′) sense: CCAGCAUCCUGACCAUCCAtt (SEQ ID NO: 1); Sequence (5′->3′) antisense: UGGAUGGUCAGGAUGCUGGag (SEQ ID NO: 2)) or control scrambled siRNA were administered locally via subconjunctival injection on Day 1 after laser.

### Laser Photocoagulation

In vivo data were obtained using a well-established mouse model of glaucoma with laser-induced occlusion of episcleral veins in normal eyes (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Briefly, mice were randomized to receive unilateral laser photocoagulation (532 nm, OcuLight TX; IRIDEX Corporation, Mountain View, CA, USA) on the right eyes. The left unlasered eyes with normal IOP were used as control for the procedure.

### Measurement of Intraocular Pressure (IOP)

Intraocular pressure (IOP) was measured by a noninvasive TonoLab tonometer (Icare Lab, Helsinki, Finland) under light general anesthesia with 2% isoflurane.

### Optical Coherence Tomography (OCT) to Measure Central Corneal Thickness

Central corneal thickness was measured using Optical Coherence Tomography (OCT) according to previously reported methods (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Quadrant-scans along four axes were performed to scan through the central cornea, while data along the 0°-180° axis were used for analysis.

### Results

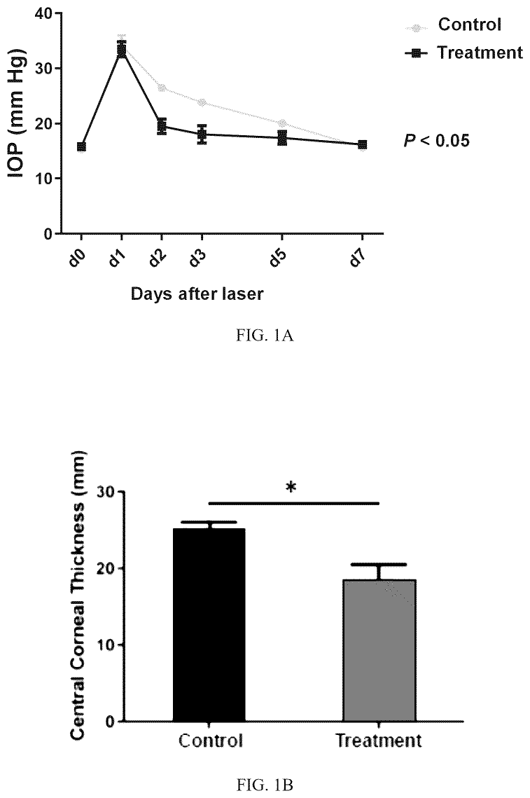

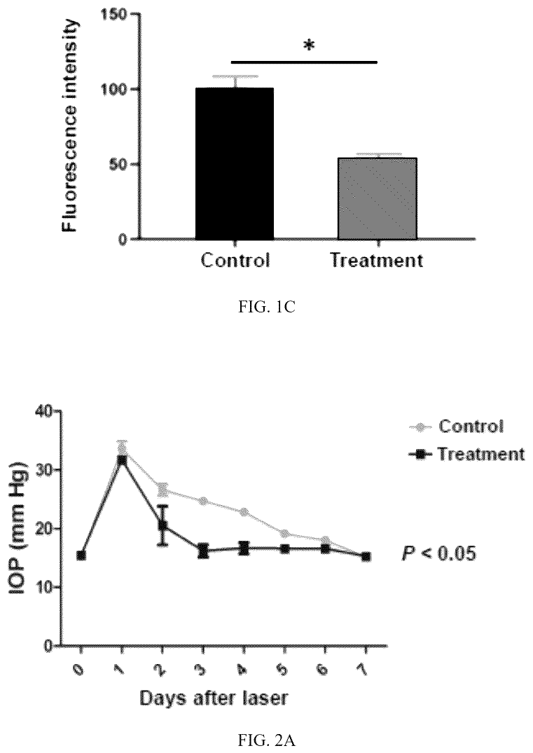

As shown in FIG. 1A, compared to the control condition, IOP in the VEGFR-3 specific siRNA treated eyes was significantly reduced after the treatment. This effect is specific since treatment with a scrambled sequence siRNA had no effect on IOP. VEGFR-3 specific siRNA treatment significantly reduced corneal edema, as measured in vivo by OCT (FIG. 1B). As shown in FIG. 1C, the summarized data with human Schlemm's canal cells shows that anti-VEGFR-3 siRNA treatment inhibited Schlemm canal cell function, such as adhesion (*P<0.05).

### Conclusion

While not wanting to be held to one theory, it is proposed that inhibiting the major pro-lymphatic formation factor VEGFR-3 targets certain lymphatic functions of the Schlemm's canal to increase the permeability thereof, which contribute to enhanced aqueous humor movement via the Schlemm's canal that provides measurable decreases in IOP and a reduction in glaucomatous damage.

### Example 2. VLA-1 Blockade Lowers IOP and Protects the Eye from Glaucomatous Damage

This example shows that the anti-VLA-1 antibody treatment significantly lowered IOP and protected the ocular tissues from glaucomatous damages, as demonstrated in a mouse model of glaucoma.

### Methods

### Materials

Intraocular hypertension was induced in normal eyes by laser. Anti-VLA-1 antibody (Clone Ha31/8) or control were administered locally via subconjunctival injection and started on Day 1 after laser.

### Laser Photocoagulation

In vivo data were obtained using a well-established mouse model of glaucoma with laser-induced occlusion of episcleral veins in normal eyes (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Briefly, mice were randomized to receive unilateral laser photocoagulation (532 nm, OcuLight TX; IRIDEX Corporation, Mountain View, CA, USA) on the right eyes. The left unlasered eyes with normal IOP were used as control for the procedure.

### Measurement of Intraocular Pressure (IOP)

Intraocular pressure (IOP) was measured by a noninvasive TonoLab tonometer (Icare Lab, Helsinki, Finland) under light general anesthesia with 2% isoflurane.

### Optical Coherence Tomography (OCT) to Measure Central Corneal Thickness and Retinal Nerve Fiber Layer Thickness

Central corneal thickness was measured using Optical Coherence Tomography (OCT) according to previously reported methods (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Quadrant-scans along four axes were performed to scan through the central cornea, while data along the 0°-180° axis were used for analysis.

For the measurement of retinal nerve fiber layer (RNFL) thickness, pupils were dilated with 1% tropicamide ophthalmic solution and retinal cross-section images were captured by OCT. A rectangular scanning sequence produced a single en face image of the retina. RNFL thickness was measured in 4 quadrants of the en face image.

### Measurement of Relative Retinal Ganglion Cell (RGC) Density

The relative number of retinal ganglion cells (RGC) were assessed as previously described (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Briefly, whole-mount retinae were harvested 7 days post-laser administration, fixed in methanol, and immunostained for Brn3a antibody. Samples were imaged and the percentage scores were obtained by normalizing to normal control condition defined as being 100%.

### Results

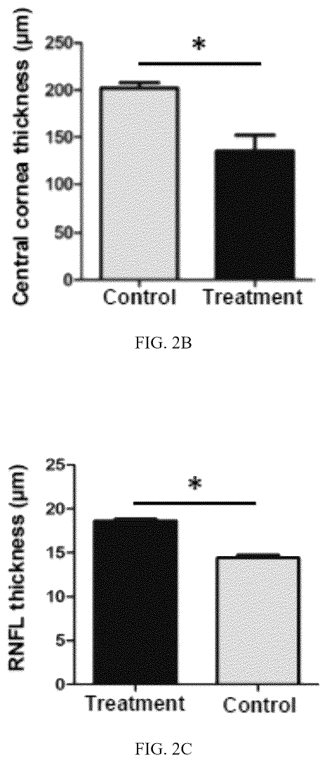

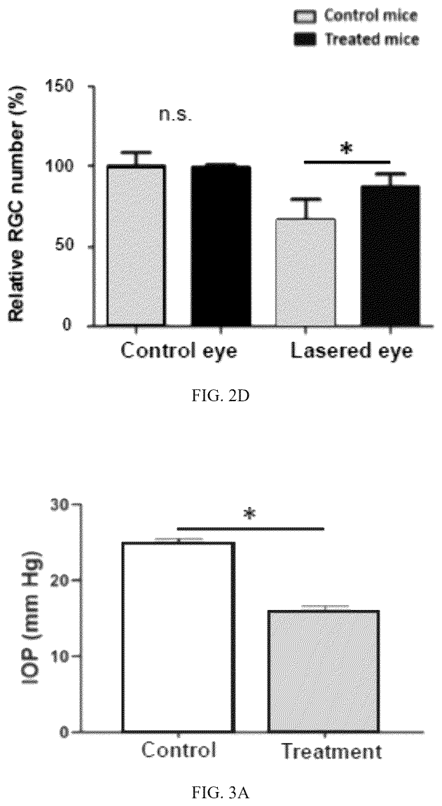

Compared to the control condition, IOP in the anti-VLA-1 antibody treated eyes was significantly reduced (FIG. 2A). The summarized data shows that anti-VLA-1 antibody treatment protected the cornea from edema (FIG. 2B) and reduced RNFL (retinal nerve fiber layer) thinning (FIG. 2C) and retinal ganglion cell (RGC) death (FIG. 2D) as well. Central corneal thickness and RNFL thickness were measured in vivo by OCT (*P<0.05; n.s. not significant).

### Example 3. VLA-1 Blockade Lowers IOP and Inhibits Schlemm's Canal Cell Function

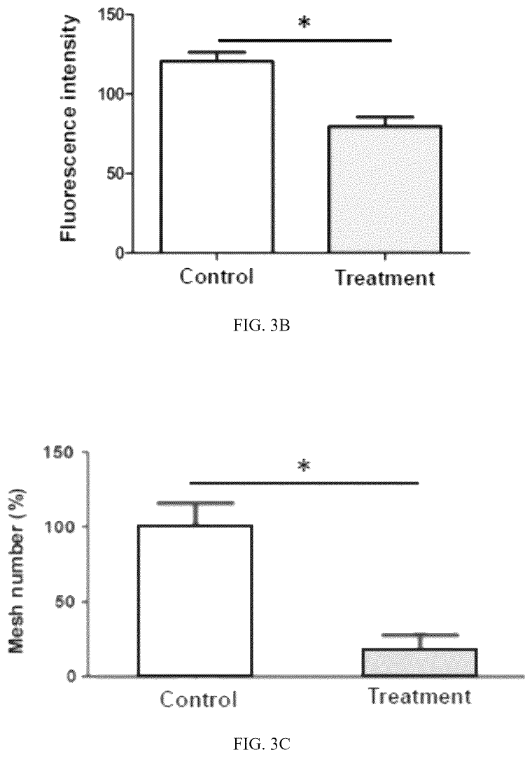

This example shows that the anti-VLA-1 treatment via siRNAs significantly lowered IOP, as demonstrated in a mouse model of glaucoma (FIG. 3A).

### Methods

### Materials

Intraocular hypertension was induced in normal eyes by laser. Two sets of anti-VLA-1 siRNA were used. To test for IOP in vivo, the following anti-VLA-1 siRNA set was used: (Sequence (5′->3′) sense: GGAUCAACUUUAGUCACCAtt (SEQ ID NO: 9); Sequence (5′->3′) antisense: UGGUGACUAAAGUUGAUCCaa (SEQ ID NO: 10)). The anti-VLA-1 siRNA or control scrambled siRNA were administered locally via subconjunctival injection on Day 1 after laser. To test for Schlemm's canal cell function in vitro, the following anti-VLA-1 siRNA set was used: (Sequence (5′->3′) sense: GGACUUUAAUCUUACCGAUtt (SEQ ID NO: 5); Sequence (5′->3′) antisense: AUCGGUAAGAUUAAAGUCCaa (SEQ ID NO: 6)).

### Laser Photocoagulation

In vivo data were obtained using a well-established mouse model of glaucoma with laser-induced occlusion of episcleral veins in normal eyes (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Briefly, mice were randomized to receive unilateral laser photocoagulation (532 nm, OcuLight TX; IRIDEX Corporation, Mountain View, CA, USA) on the right eyes. The left unlasered eyes with normal IOP were used as control for the procedure.

### Measurement of Intraocular Pressure (IOP)

Intraocular pressure (IOP) was measured by a noninvasive TonoLab tonometer (Icare Lab, Helsinki, Finland) under light general anesthesia with 2% isoflurane.

### Schlemm's Canal Cell Adhesion Assay

The Schlemm's canal cell adhesion assay was performed according to previously reported methods (Altiok, E. et al. Integrin Alpha-9 Mediates Lymphatic Valve Formation in Corneal Lymphangiogenesis. Invest Ophthalmol Vis Sci. 56 (11): 6313-6319 (2015 October)). Briefly, forty-eight hours following siRNA transfection with either an anti-VLA-1 or control scrambled siRNA, Schlemm's canal cells were seeded onto plates coated with collagen IV. Plates were incubated for 30 min at 37° C., washed with PBS, and incubated with calcein (1 ug/mL) for 30 min at room temperature. Plates were then washed with PBS and fluorescence intensity was measured with a microplate reader.

### Schlemm's Canal Cell Tube Formation Assay

The Schlemm's canal cell tube formation assay was performed according to previously reported methods (Altiok, E. et al. Integrin Alpha-9 Mediates Lymphatic Valve Formation in Corneal Lymphangiogenesis. Invest Ophthalmol Vis Sci. 56 (11): 6313-6319 (2015 October)). Briefly, forty-eight hours following siRNA transfection with either an anti-VLA-1 or control scrambled siRNA, Schlemm's canal cells were seeded onto plates containing solidified Matrigel. Tube formation was imaged at 6 hours post seeding by an inverted microscope.

### Results

IOP was measured on Day 3 after laser and it was significantly reduced in the anti-VLA-1 siRNA treated eyes compared to the control condition. This effect is specific since treatment with a scrambled sequence siRNA had no effect on IOP. FIG. 3B and FIG. 3C show the summarized data with human Schlemm's canal cells showing that anti-VLA-1 siRNA treatment inhibited Schlemm's canal cell functions, such as adhesion (FIG. 3B) and tube formation (FIG. 3C) (*P<0.05).

### Conclusion

While not wanting to be held to one theory, it is proposed that inhibiting the major pro-lymphatic formation factor VLA-1 targets certain lymphatic functions of the Schlemm's canal to increase the permeability thereof, which contribute to enhanced aqueous humor movement via the Schlemm's canal that provides measurable decreases in IOP.

### Example 4. Ang-2 Blockade Lowers IOP and Inhibits Schlemm's Canal Cell Function

This example shows that anti-Ang-2 treatment via siRNAs significantly lowered IOP, as demonstrated in a mouse model of glaucoma.

### Methods

### Materials

Intraocular hypertension was induced in normal eyes by laser. Two sets of anti-Ang-2 siRNA were used. To test for IOP in vivo, the following anti-Ang-2 siRNA set was used: (Sequence (5′->3′) sense: CCUCAGGAAUGAAUCAGAAtt (SEQ ID NO: 17); Sequence (5′->3′) antisense: UUCUGAUUCAUUCCUGAGGIA (SEQ ID NO: 18)). The anti-Ang-2 siRNA or control scrambled siRNA were administered locally via subconjunctival injection on Day 1 after laser. To test for Schlemm's canal cell function in vitro, the following anti-Ang-2 siRNA set was used: (Sequence (5′->3′) sense: GCAGAGAGAUGAAGAUCUAtt (SEQ ID NO: 15); Sequence (5′->3′) antisense: UAGAUCUUCAUCUCUCUGCaa (SEQ ID NO: 16)).

### Laser Photocoagulation

In vivo data were obtained using a well-established mouse model of glaucoma with laser-induced occlusion of episcleral veins in normal eyes (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Briefly, mice were randomized to receive unilateral laser photocoagulation (532 nm, OcuLight TX; IRIDEX Corporation, Mountain View, CA, USA) on the right eyes. The left unlasered eyes with normal IOP were used as control for the procedure.

### Measurement of Intraocular Pressure (IOP)

Intraocular pressure (IOP) was measured by a noninvasive TonoLab tonometer (Icare Lab, Helsinki, Finland) under light general anesthesia with 2% isoflurane.

### Schlemm's Canal Cell Adhesion Assay

The Schlemm's canal cell adhesion assay was performed according to previously reported methods (Altiok, E. et al. Integrin Alpha-9 Mediates Lymphatic Valve Formation in Corneal Lymphangiogenesis. Invest Ophthalmol Vis Sci. 56 (11): 6313-6319 (2015 October)). Briefly, forty-eight hours following siRNA transfection with either an anti-Ang-2 or control scrambled siRNA, Schlemm's canal cells were seeded onto plates coated with collagen IV. Plates were incubated for 30 min at 37° C., washed with PBS, and incubated with calcein (1 ug/mL) for 30 min at room temperature. Plates were then washed with PBS and fluorescence intensity was measured with a microplate reader.

### Schlemm's Canal Cell Tube Formation Assay

The Schlemm's canal cell tube formation assay was performed according to previously reported methods (Altiok, E. et al. Integrin Alpha-9 Mediates Lymphatic Valve Formation in Corneal Lymphangiogenesis. Invest Ophthalmol Vis Sci. 56 (11): 6313-6319 (2015 October)). Briefly, forty-eight hours following siRNA transfection with either an anti-Ang-2 or control scrambled siRNA, Schlemm's canal cells were seeded onto plates containing solidified Matrigel. Tube formation was imaged at 6 hours post seeding by an inverted microscope.

### Results

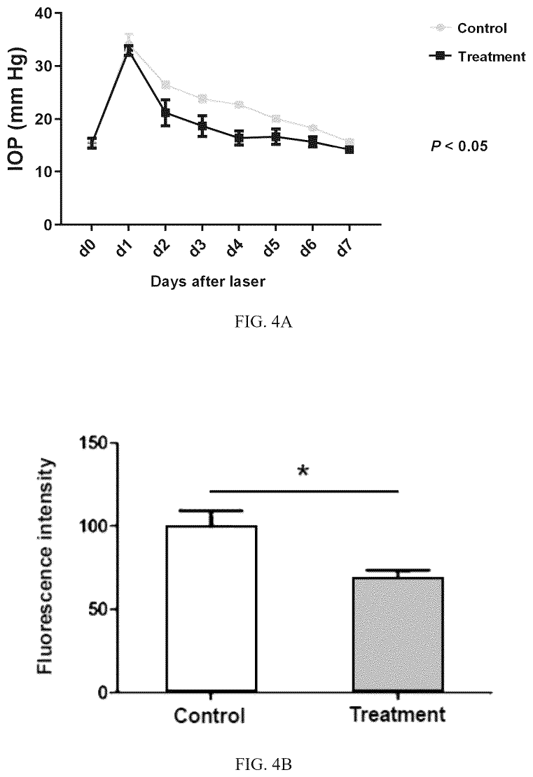

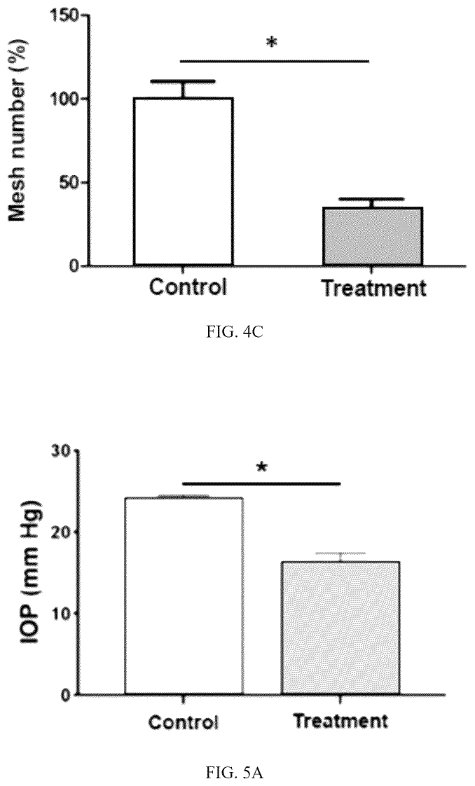

Compared to the control condition, IOP in the anti-Ang-2 siRNA treated eyes was significantly reduced (FIG. 4A). This effect is specific since treatment with a scrambled sequence siRNA had no effect on IOP. Summarized data with human Schlemm's canal cells showing that anti-Ang-2 siRNA treatment inhibited Schlemm canal cell functions, such as adhesion (FIG. 4B) and tube formation (FIG. 4C) (*P<0.05).

### Conclusion

While not wanting to be held to one theory, it is proposed that inhibiting the major pro-lymphatic formation factor Ang-2 targets certain lymphatic functions of the Schlemm's canal to increase the permeability thereof, which contribute to enhanced aqueous humor movement via the Schlemm's canal that provides measurable decreases in IOP.

### Example 5. ITGA5 Blockade Lowers IOP and Inhibits Schlemm's Canal Cell Function

This example shows that the anti-ITGA5-1 treatment via siRNAs significantly lowered IOP, as demonstrated in a mouse model of glaucoma.

### Methods

### Materials

Intraocular hypertension was induced in normal eyes by laser. Two sets of anti-ITGA5 siRNA were used. To test for IOP in vivo, the following anti-ITGA5 siRNA set was used: (Sequence (5′->3′) sense: CCCUUAUGGACGAAAUUUAtt (SEQ ID NO: 13); Sequence (5′->3′) antisense: UAAAUUUCGUCCAUAAGGGct (SEQ ID NO: 14)). Anti-ITGA5 siRNA or control scrambled siRNA were administered locally via subconjunctival injection. To test for Schlemm's canal cell function in vitro, the following anti-ITGA5 siRNA set was used: (Sequence (5′->3′) sense: CCUUCCAACUUGAACGGAAtt (SEQ ID NO: 11); Sequence (5′->3′) antisense: UUCCGUUCAAGUUGGAAGGac (SEQ ID NO: 12)).

### Laser Photocoagulation

In vivo data were obtained using a well-established mouse model of glaucoma with laser-induced occlusion of episcleral veins in normal eyes (Zhang, L. et al. Establishment and Characterization of an Acute Model of Ocular Hypertension by Laser-Induced Occlusion of Episcleral Veins. Invest Ophthalmol Vis Sci. 58 (10): 3879-3886 (2017 Aug. 1)). Briefly, mice were randomized to receive unilateral laser photocoagulation (532 nm, OcuLight TX; IRIDEX Corporation, Mountain View, CA, USA) on the right eyes. The left unlasered eyes with normal IOP were used as control for the procedure.

### Measurement of Intraocular Pressure (IOP)

Intraocular pressure (IOP) was measured by a noninvasive TonoLab tonometer (Icare Lab, Helsinki, Finland) under light general anesthesia with 2% isoflurane.

### Schlemm's Canal Cell Adhesion Assay

The Schlemm's canal cell adhesion assay was performed as described previously (Altiok, E. et al. Integrin Alpha-9 Mediates Lymphatic Valve Formation in Corneal Lymphangiogenesis. Invest Ophthalmol Vis Sci. 56 (11): 6313-6319 (2015 October)). Briefly, forty-eight hours following siRNA transfection with either an anti-pro-lymphatic formation factor or control scrambled siRNA, Schlemm's canal cells were seeded onto plates coated with collagen IV. Plates were incubated for 30 min at 37° C., washed with PBS, and incubated with calcein (1 μg/mL) for 30 min at room temperature. Plates were then washed with PBS and fluorescence intensity was measured with a microplate reader.

### Results

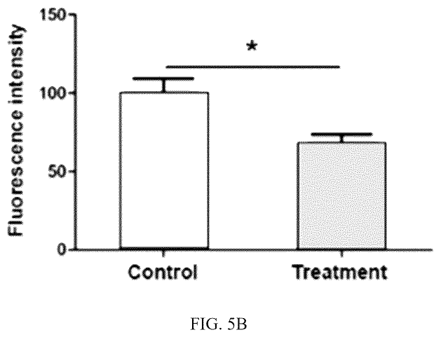

IOP was measured on Day 3 after laser and it was significantly reduced in the anti-ITGA5 siRNA treated eyes compared to the control condition (FIG. 5A). This effect is specific since treatment with a scrambled sequence siRNA had no effect on IOP. The summarized data with human Schlemm's canal cells shows that anti-ITGA5 siRNA treatment inhibited Schlemm canal cell function, such as adhesion (FIG. 5B) (*P<0.05).

### Conclusion

While not wanting to be held to one theory, it is proposed that inhibiting the major pro-lymphatic formation factor ITGA5 targets certain lymphatic functions of the Schlemm's canal to increase the permeability thereof, which contribute to enhanced aqueous humor movement via the Schlemm's canal that provides measurable decreases in IOP.

Various embodiments have been described herein with reference to the accompanying drawings. It will, however, be evident that various modifications and changes may be made thereto, and additional embodiments may be implemented, without departing from the broader scope of the invention as set forth in the claims that follow. Further, other embodiments will be apparent to those skilled in the art from consideration of the specification and practice of one or more embodiments of the invention disclosed herein. It is intended, therefore, that this application and the examples herein be considered as exemplary only, with a true scope and spirit of the invention being indicated by the following listing of exemplary claims.

What is claimed is:

1. A method for treating glaucoma in an eye of a subject in need thereof, or
for preventing glaucoma in an eye of a subject in need thereof, or
for inhibiting lymphangiogenesis in an eye of a subject at risk for the development of glaucoma, or
for inhibiting lymphangiogenesis in an eye of a subject suffering from elevated intraocular pressure (IOP),
for increasing Schlemm's canal permeability in an eye of a subject suffering from elevated IOP, or
for inhibiting Schlemm's canal cellular activity in an eye of a subject suffering from elevated IOP, wherein the cellular activity is selected from the group consisting of the inhibition of cellular adhesion, inhibition of cellular proliferation, inhibition of cellular migration, or inhibition of tube formation, or
for increasing aqueous humor outflow in an eye's Schlemm's canal of a subject suffering from elevated IOP, or
for reducing corneal edema in an eye of a subject in need thereof, or
for reducing retinal nerve fiber layer thinning in an eye of a subject in need thereof, or
for reducing retinal ganglion cell (RGC) death in an eye of a subject in need thereof,
the method comprising administering to the eye a therapeutically effective amount of an inhibitor of a pro-lymphatic formation factor, wherein the administration of the inhibitor results in lowering IOP of the eye.

2. The method of claim 1, wherein the subject is a human, and has a primary glaucoma, primary open angle glaucoma, primary angle-closure glaucoma, primary congenital glaucoma, or primary normal tension glaucoma.

3. The method of claim 1, wherein the pro-lymphatic formation factor is:
a VEGF/VEGFR family member, selected from the group consisting of VEGF-A, VEGF-B, VEGF-C, VEGF-D, VEGF-E, PIGF, VEGFR-1, VEGFR-2, and VEGFR-3; or
an integrin family member selected from the group consisting of very late antigen-1 (VLA-1), integrin alpha 5 (ITGA5), and integrin alpha 9 (ITGA9) or
an angiopoietin family member, selected from the group consisting of angiopoietin-1 (ANGPT1), angiopoietin-2 (ANGPT2 or Ang-2), and Tie2/TEK.

4. The method of claim 1, wherein the pro-lymphatic formation factor is selected from the group consisting of:
(i) a cytokine/chemokine, wherein the cytokine or chemokine is selected from the group consisting of interleukin 8, interferon gamma, CCR7, and SLC;
(ii) an extracellular matrix protein, wherein the extracellular matrix protein is CCBE1;
(iii) a transcription factor, wherein the transcription factor is selected from the group consisting of Sox18 and Hhex;
(iv) a guidance molecule, wherein the guidance molecule is selected from the group consisting of neuropilin 2 and SEMA7A;
(v) an FGF, wherein the FGF is FGF-2;
(vi) a protein tyrosine phosphatase (PTP), wherein the PTP is PTPN14;
(vii) a member of platelet factors, wherein the platelet factor member is platelet factor 4 or (viii) a platelet-derived growth factor comprising PDGF-BB;
(ix) a LEC polarity factor, wherein the LEC polarity factor is selected from the group consisting of Celsr1, Vangl2, Pdk2, and Fat4;
(x) a member of Notch family;
(xi) ARAF, SOS1, Apelin, KIF11, REELIN, CALCRL, GJC2 (gap junction protein gamma-2), Rasip1, or FBXL7 (F-box and leucine-rich repeat protein 7); and,
(xii) a combination of any one of (i)-(xi).

5. The method of claim 1, wherein pro-lymphatic formation factor selected from the group consisting of VEGFR-3, VLA-1, ITGA5, and Ang-2.

6. The method of claim 1, wherein the inhibitor of the pro-lymphatic formation factor is selected from the group consisting of:
(i) an agent capable of disrupting the pro-lymphatic formation factor's gene, thereby eliminating or reducing expression of the pro-lymphatic formation factor;
(ii) an siRNA capable of degrading the mRNA of a pro-lymphatic formation factor, thus reducing expression of the pro-lymphatic factor;
(iii) an antagonist antibody against the pro-lymphatic formation factor which inhibits or prevents the function of the pro-lymphatic formation factor;
(iv) a small molecule capable of inhibiting or preventing the function of the pro-lymphatic formation factor; and
(v) a combination of any of (i)-(iv).

7. The method of claim 1, wherein:
the inhibitor of the pro-lymphatic formation factor is administered topically, via intracameral injection, via subconjunctival injection, via intravitreal injection, via suprachoroidal injection, via peribulbar injection, or via retrobulbar injection, and,
the inhibitor of the pro-lymphatic formation factor is formulated as eye drop, depot, bolus, inhibitor-loaded contact lens, suspension, solution, ophthalmic gel, or ointment.

8. The method of claim 1, further comprising treating the eye with a second therapy selected from the group consisting of an eye drop, an oral medication, a laser therapy, and a surgery, wherein the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

9. The method of claim 1, wherein the subject, prior to administration of the inhibitor, the subject has an elevated IOP of greater than about 21, 25 or 30 mm Hg, and following administration, the subject's IOP in the treated eye is reduced by at least about 2, 5, 10 or 15 mm Hg.

10. The method of claim 1, wherein the inhibitor is an siRNA.

11. The method of claim 1, wherein the inhibitor is an antagonist antibody.

12. The method of claim 1, wherein the inhibitor disrupts the pro-lymphatic formation factor's gene's expression.

13. A method for treating glaucoma in an eye of a subject in need thereof, or
for preventing glaucoma in an eye of a subject in need thereof, or
of inhibiting lymphangiogenesis in an eye of a subject at risk for the development of glaucoma, or
of inhibiting lymphangiogenesis in an eye of a subject suffering from elevated intraocular pressure (IOP), or
for increasing Schlemm's canal permeability in an eye of a subject suffering from elevated IOP, or
for increasing aqueous humor outflow in an eye's Schlemm's canal of a subject suffering from elevated IOP, or
for inhibiting Schlemm's canal cellular activity in an eye of a subject suffering from elevated IOP, wherein the cellular activity is selected from the group consisting of the inhibition of cellular adhesion, inhibition of cellular proliferation, inhibition of cellular migration, or inhibition of tube formation; or
for reducing corneal edema in an eye of a subject in need thereof, or
for reducing retinal nerve fiber layer (RNFL) thinning in an eye of a subject in need thereof, or
for reducing retinal ganglion cell (RGC) death in an eye of a subject in need thereof,
the method comprising administering to the eye a therapeutically effective amount of an anti-lymphatic formation factor, or an agent that activates or stimulates an anti-lymphatic formation factor, wherein the administration of the agent results in lowering the TOP of the eye.

14. The method of claim 13, wherein the subject is a human, and has a primary glaucoma, primary open angle glaucoma, primary angle-closure glaucoma, primary congenital glaucoma, or primary normal tension glaucoma.

15. The method of claim 13, wherein the agent administered is selected from the group consisting of:
(i) an anti-lymphatic formation factor;
(ii) an agent that activates the expression of the anti-lymphatic formation factor;
(iii) an agonist antibody activating the anti-lymphatic formation factor;
(iv) a microRNA or mimics inhibiting lymphatic formation, and
(v) a small molecule activating the function of the anti-lymphatic formation factor.

16. The method of claim 13, wherein the anti-lymphatic formation factor is selected from the group consisting of: microRNA184, microRNA126, miR-31, miR-181a, microRNA132, miR-194, miR-186, miR-99a, miR-92a, and miR-466, endostatin, and a VEGF family inhibitor (e.g. soluble VEGFR-2, soluble VEGFR-3).

17. The method of claim 13, wherein:
anti-lymphatic formation factor, or an agent is administered topically, via intracameral injection, via subconjunctival injection, via intravitreal injection, via suprachoroidal injection, via peribulbar injection, or via retrobulbar injection, and,
anti-lymphatic formation factor, or an agent is formulated as eye drop, depot, bolus, inhibitor-loaded contact lens, suspension, solution, ophthalmic gel, or ointment.

18. The method of claim 13, further comprising treating the eye with a second therapy selected from the group consisting of an eye drop, an oral medication, a laser therapy, and a surgery, wherein the second therapy is selected from the group consisting of an eye drop that decreases the amount of fluid produced in the eye or improves the drainage of fluid from the eye, an oral medication that lowers IOP, a laser therapy comprising laser trabeculoplasty or laser iridotomy, and a surgery comprising implants or trabeculectomy.

19. The method of claim 13, wherein the subject, prior to administration of the anti-lymphatic formation factor, or agent, the subject has an elevated IOP of greater than about 21, 25 or 30 mm Hg, and following administration, the subject's IOP in the treated eye is reduced by at least about 2, 5, 10 or 15 mm Hg.


SEQUENCE LISTING (28 sequences)

SEQ ID NO: 1 - DNA, 21 bp, synthetic construct
  misc_feature 1..19: note = RNA
  misc_feature 20..21: note = DNA
  source 1..21: mol_type = other DNA
ccagcatcctgaccatccatt

SEQ ID NO: 2 - DNA, 21 bp, synthetic construct
  misc_feature 1..19: note = RNA
  misc_feature 20..21: note = DNA
  source 1..21: mol_type = other DNA
tggatggtcaggatgctggag

SEQ ID NO: 3 - DNA, 21 bp, synthetic construct
  misc_feature 1..19: note = RNA
  misc_feature 20..21: note = DNA
  source 1..21: mol_type = other DNA
gctggttttgaactgtacatt

SEQ ID NO: 4 - DNA, 21 bp, synthetic construct
  misc_feature 1..19: note = RNA
  misc_feature 20..21: note = DNA
  source 1..21: mol_type = other DNA
tgtacagttcaaaaccagctt

SEQ ID NO: 5 - DNA, 21 bp, synthetic construct
  misc_feature 1..19: note = RNA
  misc_feature 20..21: note = DNA
  source 1..21: mol_type = other DNA
ggactttaatcttaccgattt

SEQ ID NO: 6 - DNA, 21 bp, synthetic construct
  misc_feature 1..19: note = RNA
  misc_feature 20..21: note = DNA
  source 1..21: mol_type = other DNA
atcggtaagattaaagtccaa

SEQ ID NO: 7 - DNA, 21 bp, synthetic construct
  misc_feature 1..19: note = RNA
  misc_feature 20..21: note = DNA
  source 1..21: mol_type = other DNA
gagtgaaattcattggatatt

SEQ ID NO: 8 - DNA, 21 bp, synthetic construct
  misc_feature 1..19: note = RNA
  misc_feature 20..21: note = DNA
  source 1..21: mol_type = other DNA
tatccaatgaatttcactcct

SEQ ID NO: 9 - DNA, 21 bp, synthetic construct
  misc_feature 1..19: note = RNA
  misc_feature 20..21: note = DNA
  source 1..21: mol_type = other DNA
ggatcaactttagtcaccatt

SEQ ID NO: 10 - DNA, 21 bp, synthetic construct
  misc_feature 1..19: note = RNA
  misc_feature 20..21: note = DNA
  source 1..21: mol_type = other DNA
tggtgactaaagttgatccaa

SEQ ID NO: 11 - DNA, 21 bp, synthetic construct
  misc_feature 1..19: note = RNA
  misc_feature 20..21: note = DNA
  source 1..21: mol_type = other DNA
ccttccaacttgaacggaatt

SEQ ID NO: 12 - DNA, 21 bp, synthetic construct
  misc_feature 1..19: note = RNA
  misc_feature 20..21: note = DNA
  source 1..21: mol_type = other DNA
ttccgttcaagttggaaggac

SEQ ID NO: 13 - DNA, 21 bp, synthetic construct
  misc_feature 1..19: note = RNA
  misc_feature 20..21: note = DNA
  source 1..21: mol_type = other DNA
cccttatggacgaaatttatt

SEQ ID NO: 14 - DNA, 21 bp, synthetic construct
  misc_feature 1..19: note = RNA
  misc_feature 20..21: note = DNA
  source 1..21: mol_type = other DNA
taaatttcgtccataagggct

SEQ ID NO: 15 - DNA, 21 bp, synthetic construct
  misc_feature 1..19: note = RNA
  misc_feature 20..21: note = DNA
  source 1..21: mol_type = other DNA
gcagagagatgaagatctatt

SEQ ID NO: 16 - DNA, 21 bp, synthetic construct
  misc_feature 1..19: note = RNA
  misc_feature 20..21: note = DNA
  source 1..21: mol_type = other DNA
tagatcttcatctctctgcaa

SEQ ID NO: 17 - DNA, 21 bp, synthetic construct
  misc_feature 1..19: note = RNA
  misc_feature 20..21: note = DNA
  source 1..21: mol_type = other DNA
cctcaggaatgaatcagaatt

SEQ ID NO: 18 - DNA, 21 bp, synthetic construct
  misc_feature 1..19: note = RNA
  misc_feature 20..21: note = DNA
  source 1..21: mol_type = other DNA
ttctgattcattcctgaggta

SEQ ID NO: 19 - DNA, 21 bp, synthetic construct
  misc_feature 1..19: note = RNA
  misc_feature 20..21: note = DNA
  source 1..21: mol_type = other DNA
ggacaatgatgggttcccatt

SEQ ID NO: 20 - DNA, 21 bp, synthetic construct
  misc_feature 1..19: note = RNA
  misc_feature 20..21: note = DNA
  source 1..21: mol_type = other DNA
tgggaacccatcattgtccag

SEQ ID NO: 21 - DNA, 21 bp, synthetic construct
  misc_feature 1..19: note = RNA
  misc_feature 20..21: note = DNA
  source 1..21: mol_type = other DNA
ggaacttattctactttggtt

SEQ ID NO: 22 - DNA, 21 bp, synthetic construct
  misc_feature 1..19: note = RNA
  misc_feature 20..21: note = DNA
  source 1..21: mol_type = other DNA
ccaaagtagaataagttcctt

SEQ ID NO: 23 - DNA, 21 bp, synthetic construct
  misc_feature 1..19: note = RNA
  misc_feature 20..21: note = DNA
  source 1..21: mol_type = other DNA
ccatcaacctatacatgcttt

SEQ ID NO: 24 - DNA, 21 bp, synthetic construct
  misc_feature 1..19: note = RNA
  misc_feature 20..21: note = DNA
  source 1..21: mol_type = other DNA
agcatgtataggttgatggtg

SEQ ID NO: 25 - DNA, 21 bp, synthetic construct
  misc_feature 1..19: note = RNA
  misc_feature 20..21: note = DNA
  source 1..21: mol_type = other DNA
gagaaaaaccggaaagagatt

SEQ ID NO: 26 - DNA, 21 bp, synthetic construct
  misc_feature 1..19: note = RNA
  misc_feature 20..21: note = DNA
  source 1..21: mol_type = other DNA
tctctttccggtttttctcag

SEQ ID NO: 27 - RNA, 20 nt, synthetic construct
  source 1..20: mol_type = other RNA
tcacagaagtaaaggagaaa

SEQ ID NO: 28 - RNA, 21 nt, synthetic construct
  source 1..21: mol_type = other RNA
aagaagaaagtcgtactatag